(19) United States
(12) Patent Application Publication
(10) Pub. No.: US 2026/0265214 A1
(43) Pub. Date: Sep. 10, 2026

(54) NOVEL DERIVATIVES OF GLYCEROL

(71) Applicant: SYMRISE AG, Holzminden (DE)

(72) Inventors: Philip KRAFT, Holzminden (DE); Lara-Joy KLEINE-BENNE, Holzminden (DE); Bernd HÖLSCHER, Hameln (DE); Patrick WARNEKE, Höxter (DE)

(73) Assignee: SYMRISE AG, Holzminden (DE)

(21) Appl. No.: 19/168,050

(22) PCT Filed: May 30, 2023
(86) PCT No.: PCT/EP2023/064366

§ 371 Date: Sep. 23, 2025

(51) Int. Cl. C07D 317/22; C07D 317/24; C11B 9/00

(52) U.S. Cl. CPC C07D 317/22; C07D 317/24; C11B 9/0076

(57) ABSTRACT

The present invention relates to a compound of formula (I) as defined herein. The present invention further relates to a mixture comprising or consisting of a compound of formula (Ia) and a compound of formula (Ib) as defined herein, wherein the compound of formula (Ia) and the compound of formula (Ib) are compounds with identical constitutional formulae. It also relates to methods for manufacturing a compound of formula (I) and/or a compound of formula (VIII) as defined herein. Moreover, the present invention relates to a compound of formula (VIII) as defined herein and to the use of a compound of formula (I) and/or of a compound of formula (VIII) as defined herein as fragrance and/or aroma substance(s). Finally, the present invention relates to fragrance and/or aroma compositions and products as defined herein, methods for manufacturing a product as defined herein, and methods for providing and/or enhancing a transparent soft white-floral jasminic note to/in a product.

The present invention relates to a compound of formula (I) as defined herein. The present invention further relates to a mixture comprising or consisting of a compound of formula (Ia) and a compound of formula (Ib) as defined herein, wherein the compound of formula (Ia) and the compound of formula (Ib) are compounds with identical constitutional formulae. It also relates to methods for manufacturing a compound of formula (I) and/or a compound of formula (VIII) as defined herein. Moreover, the present invention relates to a compound of formula (VIII) as defined herein and to the use of a compound of formula (I) and/or of a compound of formula (VIII) as defined herein as fragrance and/or aroma substance(s). Finally, the present invention relates to fragrance and/or aroma compositions and products as defined herein, methods for manufacturing a product as defined herein, and methods for providing and/or enhancing a transparent soft white-floral jasminic note to/in a product.

Further aspects of the present invention will arise from the description below, in particular from the examples, as well as from the attached patent claims.

With its transparent floral-jasminic note accompanied by the soft-fruity freshness of lemons, methyl dihydrojasmonate (methyl 2-(3′-oxo-2′-pentylcyclopentyl)acetate, Hedione®, CAS No. 24851-98-7, RIFM ID 850) creates the imaginary aura of a sun-blessed lemon on a windowsill. First obtained by hydrogenation in the course of the structure elucidation of methyl jasmonate, isolated from Egyptian jasmine concrete in 1961 by Edouart Demole (G. Ohloff, W. Pickenhagen, P. Kraft, F. Grau, Scent and Chemistry—The Molecular World of Odors, Wiley-VCH, Weinheim, 2022, p. 243), methyl dihydrojasmonate has become a true phenomenon because of its efficiency and versatility in perfume compositions. It already imparts floral transparency in trace amounts of <1%, but can also be used as main component in amounts of >20% without overpowering the whole perfume composition. On its own, the odor of methyl dihydrojasmonate is rather subtle and faint, and thus it was allegedly missed by several fragrance companies. It was Edmond Roudnitska with “Eau Sauvage” (Dior, 1966, containing less than 2% methyl dihydrojasmonate) who discovered the remarkable effects of methyl dihydrojasmonate, and thereby initiated a trend for luminous transparent perfumes that endures until today. The odor of methyl dihydrojasmonate is mainly due to its cis-configured (+)-(1R,2S)-enantiomer, and thus several qualities enriched in the cis- or the (+)-(1R,2S)-isomers were introduced on the market. With a current estimated use of 6000-8000 t/year, methyl dihydrojasmonate is one of the workhorse key ingredients in perfumery creation. Despite this high volume, methyl dihydrojasmonate does not accumulate in the environment, is readily biodegradable (85% in 28 days), does not possess any significant irritation, sensitization or cross sensitization potential, and has a very low human and environmental toxicity. This makes it almost the perfect perfumery ingredient, if only it were accessible in a sustainable and cost-efficient way, preferably from biobased starting materials. But as this is not the case and the vast majority of methyl dihydrojasmonate is of petrochemical origin, except negligible quantities for flavor creations, there is an ongoing demand in the fragrance and flavor industry for new compounds that impart, enhance, or improve methyl dihydrojasmonate-like odor notes and effects.

It was thus an object of the present invention to provide new compounds that impart, enhance, or improve methyl dihydrojasmonate-like odor notes and effects.

Further objects underlying the present invention follow from the description below and the present patent claims.

According to a first aspect of the present invention, the stated object is surprisingly achieved by a compound of formula (I)

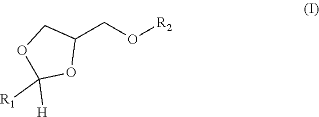

wherein
R₁ is selected from the group consisting of n-pentyl, n-hexyl, and n-heptyl, and
R₂ is selected from the group consisting of H, methyl, ethyl, n-propyl, isopropyl, allyl,

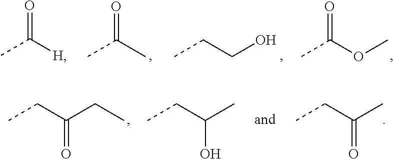

and

In the studies underlying the present invention, it was found that the compounds of formula (I) according to the present invention (or as defined herein) exhibit very similar olfactory effects to methyl dihydrojasmonate in perfume compositions (cf. the Examples section further below). This was particularly surprising since said compounds of formula (I) fall in part under a general structural motif that has been revealed in WO 2013/041130 A1 to possess fruity-green, galbanum-like odor notes. The compounds of formula (I) according to the present invention are not disclosed in WO 2013/041130 A1 and none of the compounds exemplified in said disclosure possess odor notes according to the present invention.

As will be demonstrated further below, the compounds of formula (I) are accessible not only via precursors of petrochemical origin, but advantageously also in a sustainable and/or cost-efficient way from biobased starting materials comprising only non-fossil fuel-based carbon.

Thus, according to a preferred embodiment, the compound of formula (I) comprises at least 10, 20, 30, 40, 50, 60, 70, 80, or 90 wt. %, preferably more than 50 wt. %, of biobased carbon (i.e. non-fossil fuel-based carbon), based on the total weight of the molecule. More preferably, the compound of formula (I) contains biobased carbon exclusively.

According to another preferred embodiment of the present invention, more than 50% of the carbon atoms contained in the compound of formula (I) are biobased, based on the total number of carbon atoms contained in the compound of formula (I). More preferably, more than 60%, 70%, 75%, 80%, 85%, or 90% of the carbon atoms contained in the compound of formula (I) are biobased, based on the total number of carbon atoms contained in the compound of formula (I). Most preferably, more than 75% of the carbon atoms contained in the compound of formula (I) are biobased, based on the total number of carbon atoms contained in the compound of formula (I).

Only organic compounds composed of carbon atoms from biobased sources such as plant sugars, starches or vegetal oil derivatives are considered to not further contribute to the greenhouse effect, when compared to the same organic compounds that are fossil fuel-based.

Assessment of the biobased carbon in a material can be performed through standard test methods. Using radiocarbon and isotope ratio mass spectrometry analysis, the biobased content of materials can be determined. ASTM International, formally known as the American Society for Testing and Materials, has established a standard test method for assessing the biobased content of materials. The ASTM method is designated ASTM-06866.

According to a preferred embodiment of the compound of formula (I), R₁ is either n-hexyl or n-heptyl, more preferably is n-hexyl, and/or R₂ is selected from the group consisting of methyl, ethyl, n-propyl, allyl,

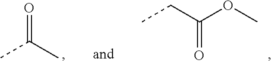

most preferably is allyl.

According to another preferred embodiment, the compound of formula (I) is selected from the group consisting of

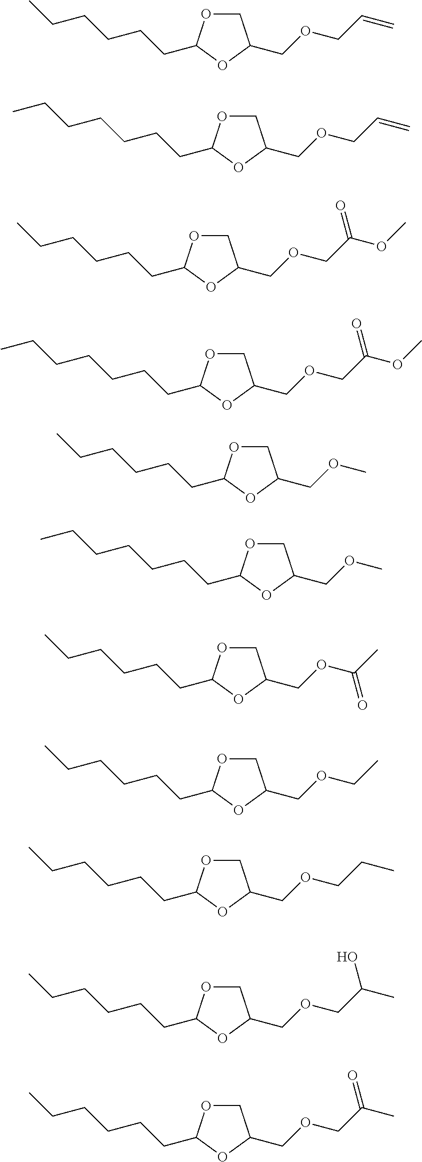

Preferably, the compound of formula (I) is a compound of formula (Ia) or a compound of formula (Ib)

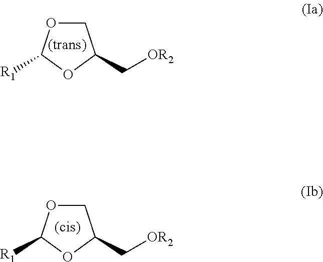

more preferably is a compound of formula (Ia), i.e. trans-configured,
wherein R₁ is selected from the group consisting of n-pentyl, n-hexyl and n-heptyl, preferably is either n-hexyl or n-heptyl, and R₂ is selected from the group consisting of methyl, ethyl, n-propyl, isopropyl, allyl,

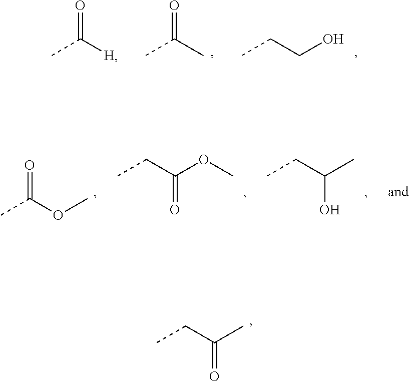

more preferably is selected from the group consisting of methyl, ethyl, n-propyl, allyl,

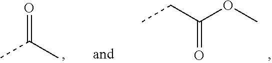

most preferably is allyl.

Another aspect of the present invention relates to a mixture comprising or consisting of a compound of formula (Ia) and a compound of formula (Ib) as defined herein, wherein the compound of formula (Ia) and the compound of formula (Ib) are compounds with identical constitutional formulae, preferably wherein the weight ratio of the compound of formula (Ia) to the compound of formula (Ib) is in a range of from 90:10 to 99:1, more preferably from 65:35 to 99:1, most preferably from 50:50 to 99:1.

In the studies underlying the present invention, it was surprisingly, and different than for methyl dihydrojasmonate, found that the compounds of formula (Ia), i.e. the trans-configured isomers, have particularly advantageous olfactory properties. Mixtures according to the invention containing a larger amount of compound of formula (Ia) than compound of formula (Ib) as defined herein are thus preferred.

According to a preferred embodiment, the compound of formula (Ia) is selected from the group consisting of

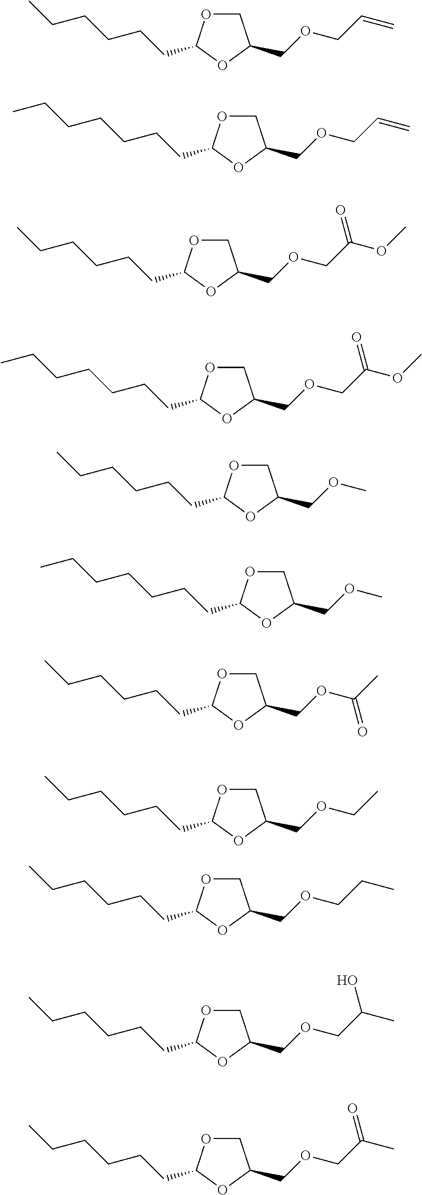

According to another preferred embodiment, the compound of formula (Ib) is selected from the group consisting of

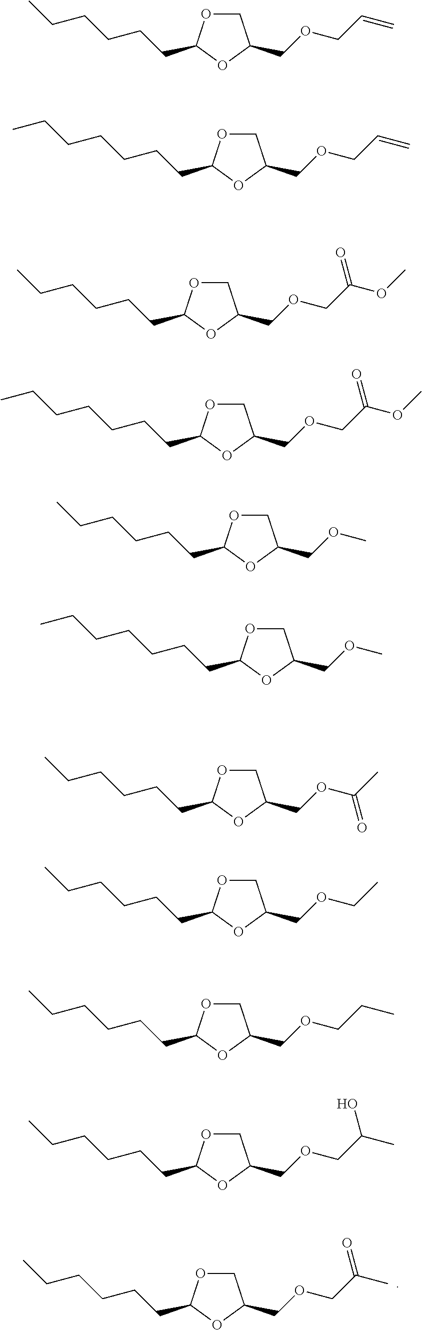

Another aspect of the present invention relates to a method for manufacturing a compound of formula (I) as defined herein, comprising or consisting of the following step:
(i) Reacting an aldehyde of formula (II), preferably of biobased origin,

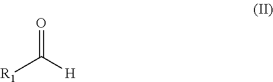

wherein R₁ is selected from the group consisting of n-pentyl, n-hexyl, and n-heptyl, preferably is either n-hexyl or n-heptyl,
with a compound of formula (III), preferably of biobased origin,

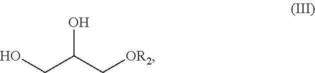

wherein R₂ is selected from the group consisting of methyl, ethyl, n-propyl, isopropyl, allyl,

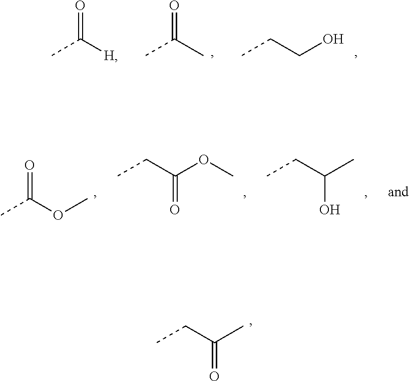

preferably is selected from the group consisting of methyl, ethyl, n-propyl, allyl,

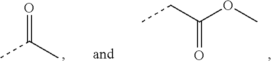

more preferably is allyl,
preferably in the presence of, for example, a (protic) acid, sodium hydrogencarbonate (NaHCO₃), or a dehydrating agent which shifts the reaction equilibrium from the hemiacetal to the acetal.

The following publications provide further details regarding the reaction conditions of the acetalization reactions according to the present invention: E. Fanghänel et al. “Organikum. Organisch-chemisches Grundpraktikum”, 24 rev. Ed., Wiley-VCH Verlag & Co. KGaA, Weinheim, 2015, pp. 480-484, and Clayden, Greeves, Warren and Wothers, Organic Chemistry, Oxford University Press, Oxford, 2001, pp. 342-347.

According to a preferred embodiment, the compound of formula (III), which is reacted in step (i) of the method according to the present invention, is obtained or obtainable according to the following steps from allyl alcohol as a starting material:

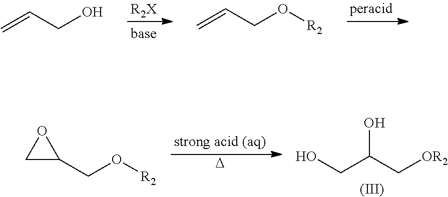

wherein X is selected from the group consisting of —Cl, —Br, and —I, preferably is —Br or —I,
and R₂ is selected from the group consisting of methyl, ethyl, n-propyl, isopropyl, allyl,

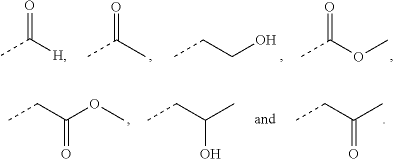

preferably is selected from the group consisting of methyl, ethyl, n-propyl, allyl,

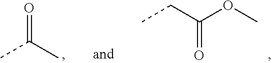

more preferably is allyl.

An example of a peracid, that may be used in the reaction, is peracetic acid and an example of a strong aqueous acid, that may be used in the reaction, is perchloric acid.

Allyl chloride can be used as a starting material to access the allyl alcohol. Allyl chloride can be reacted with sodium acetate to give allyl acetate, which can then be hydrolyzed (for instance with sodium hydroxide) to obtain the allyl alcohol.

Another aspect of the present invention relates to a method for manufacturing a compound of formula (I) as defined herein and/or a compound of formula (VIII)

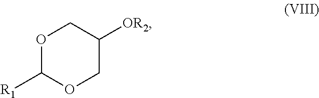

wherein
R₁ is selected from the group consisting of n-pentyl, n-hexyl, and n-heptyl, preferably is either n-hexyl or n-heptyl,
and
R₂ is selected from the group consisting of methyl, ethyl, n-propyl, isopropyl, allyl,

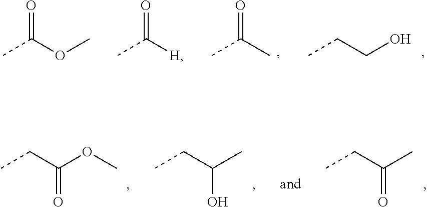

preferably is selected from the group consisting of methyl, ethyl, n-propyl, allyl,

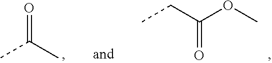

more preferably is allyl,
comprising or consisting of the following steps:
(i) Reacting an aldehyde of formula (II), preferably of biobased origin,

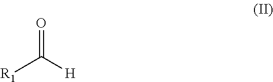

wherein R₁ is selected from the group consisting of n-pentyl, n-hexyl and n-heptyl, preferably is either n-hexyl or n-heptyl,
with glycerol, preferably of biobased origin,

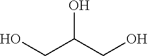

to obtain a compound of formula (IV) and/or a compound of formula (V),

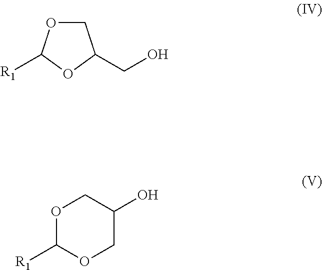

wherein R₁ is selected from the group consisting of n-pentyl, n-hexyl, and n-heptyl, preferably is either n-hexyl or n-heptyl, and
(ii) reacting the compound(s) of formula (IV) and/or formula (V) obtained in step (i) with
   a compound of formula (VI)

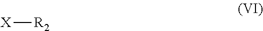

wherein X is selected from the group consisting of —Cl, —Br, and —I, and R₂ is selected from the group consisting of methyl, ethyl, n-propyl, isopropyl, allyl,

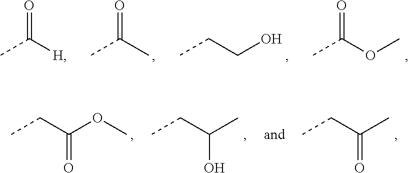

preferably wherein R₂ is selected from the group consisting of methyl, ethyl, n-propyl, allyl,

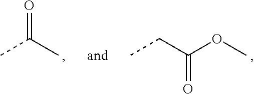

more preferably wherein R₂ is allyl,
most preferably wherein X is —Cl and R₂ is allyl (i.e. the compound of formula (VI) is 3-chloroprop-1-ene),
and/or, preferably or,
   a compound of formula (VI)

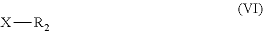

wherein X is —OH and R₂ is selected from the group consisting of

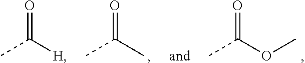

(i.e. the compound of formula (VI) is formic acid or acetic acid),
and/or, preferably or,
   a compound of formula (VII)

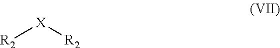

wherein X is —O— and R₂ is selected from the group consisting of

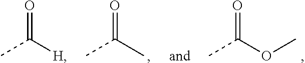

and optionally
(iii) hydrogenation of one or more carbon-carbon double bonds, if present, of the compound(s) obtained in step (ii) to carbon-carbon single bonds, preferably with hydrogen gas in the presence of palladium on charcoal as a catalyst.

Preferably, the method according to the invention is a method for manufacturing a compound of formula (I) as defined herein, comprising or consisting of the following steps:
(i) Reacting an aldehyde of formula (II), preferably of biobased origin,

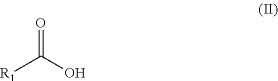

wherein R₁ is selected from the group consisting of n-pentyl, n-hexyl and n-heptyl, preferably is either n-hexyl or n-heptyl,
with glycerol, preferably of biobased origin,

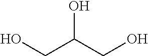

to obtain a compound of formula (IV) and/or a compound of formula (V), preferably to obtain a compound of formula (IV),

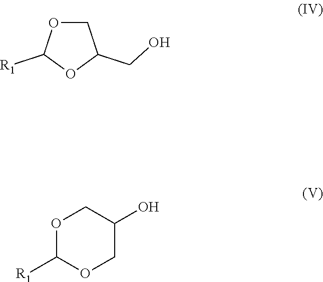

wherein R₁ is selected from the group consisting of n-pentyl, n-hexyl, and n-heptyl, preferably is either n-hexyl or n-heptyl, and
(ii) reacting the compound(s) of formula (IV) and/or formula (V), preferably the a compound of formula (IV), obtained in step (i) with
   a compound of formula (VI)

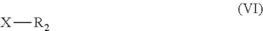

wherein X is selected from the group consisting of —Cl, —Br, and —I, and R₂ is selected from the group consisting of methyl, ethyl, n-propyl, isopropyl, allyl,

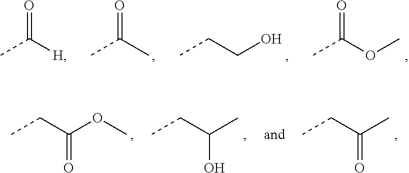

preferably wherein R₂ is selected from the group consisting of methyl, ethyl, n-propyl, allyl,

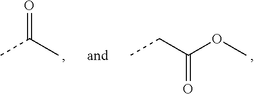

more preferably wherein R₂ is allyl,
most preferably wherein X is —Cl and R₂ is allyl (i.e. the compound of formula (VI) is 3-chloroprop-1-ene),
and/or, preferably or,
   a compound of formula (VI)

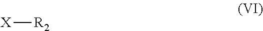

wherein X is —OH and R₂ is selected from the group consisting of

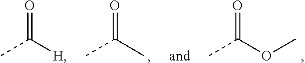

(i.e. the compound of formula (VI) is formic acid or acetic acid),
and/or, preferably or,
   a compound of formula (VII)

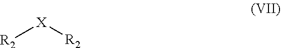

wherein X is —O— and R₂ is selected from the group consisting of

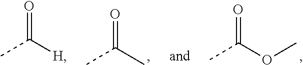

and optionally
(iii) hydrogenation of one or more carbon-carbon double bonds, if present, of the compound(s) obtained in step (ii) to carbon-carbon single bonds, preferably with hydrogen gas in the presence of palladium on charcoal as a catalyst.

Preferably, the method according to the invention is a method for manufacturing a compound of formula (VIII)

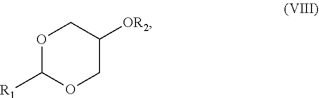

wherein
R₁ is selected from the group consisting of n-pentyl, n-hexyl, and n-heptyl, preferably is either n-hexyl or n-heptyl,
and
R₂ is selected from the group consisting of methyl, ethyl, n-propyl, isopropyl, allyl,

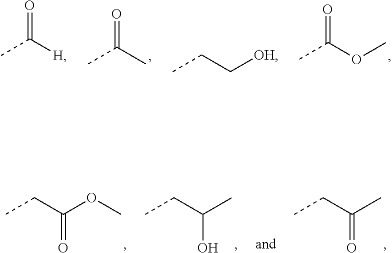

preferably is selected from the group consisting of methyl, ethyl, n-propyl, allyl,

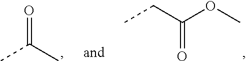

more preferably is allyl,
comprising or consisting of the following steps:
(i) Reacting an aldehyde of formula (II), preferably of biobased origin,

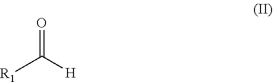

wherein R₁ is selected from the group consisting of n-pentyl, n-hexyl and n-heptyl, preferably is either n-hexyl or n-heptyl,
with glycerol, preferably of biobased origin,

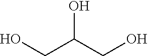

to obtain a compound of formula (IV) and/or a compound of formula (V), preferably to obtain a compound of formula (V),

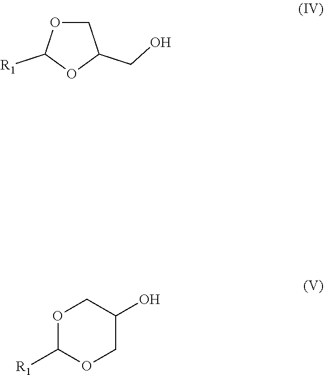

wherein R₁ is selected from the group consisting of n-pentyl, n-hexyl, and n-heptyl, preferably is either n-hexyl or n-heptyl, and
(ii) reacting the compound(s) of formula (IV) and/or formula (V), preferably the compound of formula (V), obtained in step (i) with
   a compound of formula (VI)

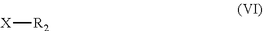

wherein X is selected from the group consisting of —Cl, —Br, and —I, and R₂ is selected from the group consisting of methyl, ethyl, n-propyl, isopropyl, allyl,

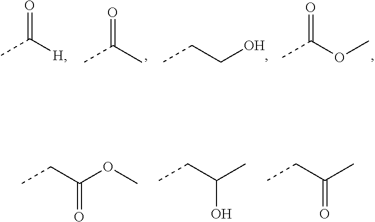

and
preferably wherein R₂ is selected from the group consisting of methyl, ethyl, n-propyl, allyl,

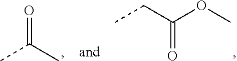

more preferably wherein R₂ is allyl,
most preferably wherein X is —Cl and R₂ is allyl (i.e. the compound of formula (VI) is 3-chloroprop-1-ene),
and/or, preferably or,
   a compound of formula (VI)

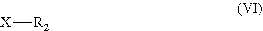

wherein X is —OH and R₂ is selected from the group consisting of

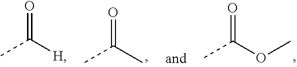

(i.e. the compound of formula (VI) is formic acid or acetic acid),
and/or, preferably or,
   a compound of formula (VII)

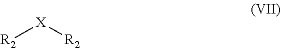

wherein X is —O— and R₂ is selected from the group consisting of

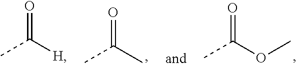

and optionally
(iii) hydrogenation of one or more carbon-carbon double bonds, if present, of the compound(s) obtained in step (ii) to carbon-carbon single bonds, preferably with hydrogen gas in the presence of palladium on charcoal as a catalyst.

Another aspect of the present invention relates to a compound of formula (VIII)

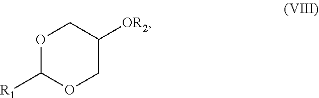

wherein
R₁ is selected from the group consisting of n-pentyl, n-hexyl, and n-heptyl, preferably is either n-hexyl or n-heptyl,
and
R₂ is selected from the group consisting of H, methyl, ethyl, n-propyl, isopropyl, allyl,

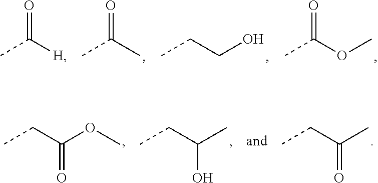

In the studies underlying the present invention, it was surprisingly found that the compounds of formula (VIII) according to the present invention (or as defined herein) exhibit very similar olfactory effects to methyl dihydrojasmonate in perfume compositions (cf. Examples in the section further below).

As shown above, the compounds of formula (I) and the compounds of formula (VIII) are accessible via the same synthetic pathway by reacting an aldehyde of formula (II) as defined herein with (unprotected) glycerol and subsequently functionalizing the remaining hydroxyl group with an R₂ residue as defined herein.

Advantageously, the compounds of formula (VIII) are accessible not only via precursors of petrochemical origin, but also in a sustainable and/or cost-efficient way from biobased starting materials comprising only non-fossil fuel-based carbon (as will be demonstrated further below).

Thus, according to a preferred embodiment, the compound of formula (VIII) comprises at least 10, 20, 30, 40, 50, 60, 70, 80, or 90 wt. %, preferably more than 50 wt. %, of biobased carbon (i.e. non-fossil fuel-based carbon), based on the total weight of the molecule. More preferably, the compound of formula (VIII) contains biobased carbon exclusively.

According to another preferred embodiment of the present invention, more than 50% of the carbon atoms contained in the compound of formula (VIII) are biobased, based on the total number of carbon atoms contained in the compound of formula (VIII). More preferably, more than 60%, 70%, 75%, 80%, 85%, or 90% of the carbon atoms contained in the compound of formula (VIII) are biobased, based on the total number of carbon atoms contained in the compound of formula (VIII). Most preferably, more than 75% of the carbon atoms contained in the compound of formula (VIII) are biobased, based on the total number of carbon atoms contained in the compound of formula (VIII).

According to a preferred embodiment of the compound of formula (VIII), R₁ is either n-hexyl or n-heptyl, and/or R₂ is selected from the group consisting of methyl, ethyl, n-propyl, allyl,

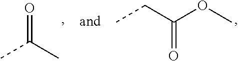

more preferably is allyl.

According to another preferred embodiment, the compound of formula (VIII) is selected from the group consisting of

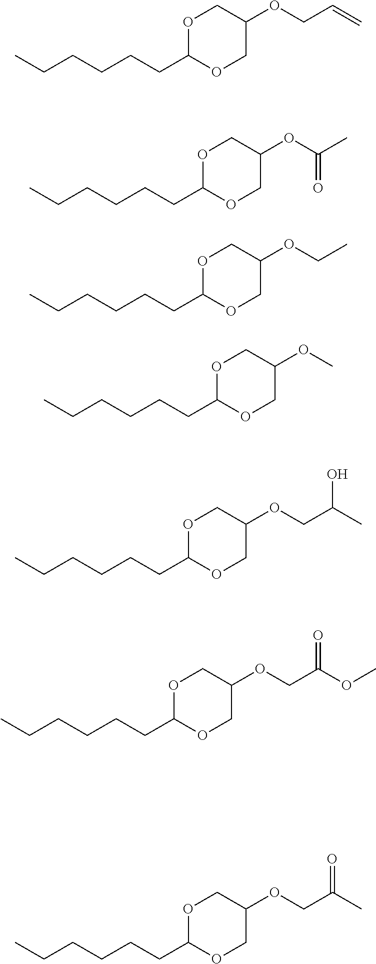

Preferably, the compound of formula (VIII) is a compound of formula (VIIIa) or a compound of formula (VIIIb)

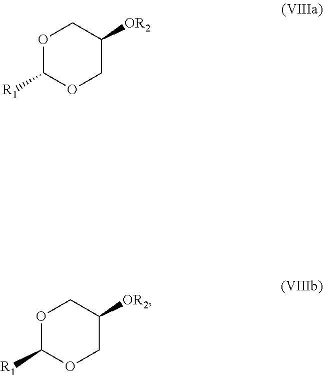

preferably is a compound of formula (VIIIa),
wherein R₁ is selected from the group consisting of n-pentyl, n-hexyl and n-heptyl, preferably is either n-hexyl or n-heptyl,
and
R₂ is selected from the group consisting of H, methyl, ethyl, n-propyl, isopropyl, allyl,

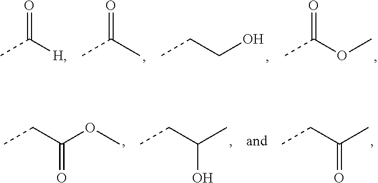

preferably is selected from the group consisting of methyl, ethyl, n-propyl, allyl,

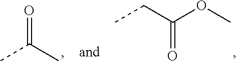

most preferably is allyl.

Another aspect of the present invention relates to a mixture comprising or consisting of a compound of formula (VIIIa) and a compound of formula (VIIIb) as defined herein, wherein the compound of formula (VIIIa) and the compound of formula (VIIIb) are compounds with identical constitutional formulae, preferably wherein the weight ratio of the compound of formula (VIIIa) to the compound of formula (VIIIb) is in a range of from 90:10 to 99:1, more preferably from 65:35 to 99:1, most preferably from 50:50 to 99:1.

In the studies underlying the present invention, it was surprisingly found that the compounds of formula (VIIIa), i.e. the trans-configured isomers, have particularly advantageous olfactory properties. Mixtures according to the invention containing a larger amount of compound of formula (VIIIa) than compound of formula (VIIIb) as defined herein are thus preferred.

According to a preferred embodiment, the compound of formula (VIIIa) is selected from the group consisting of

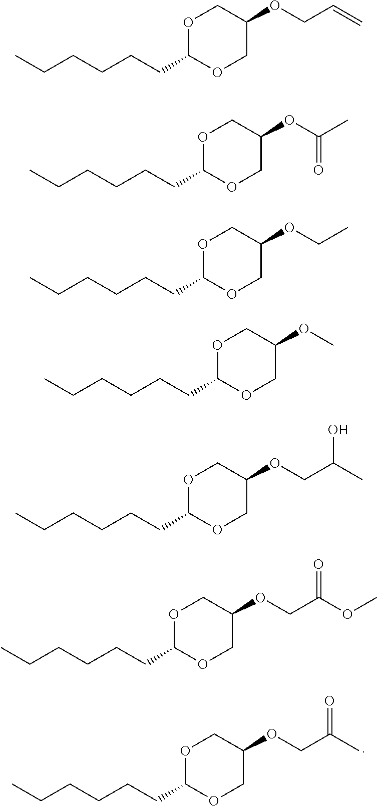

According to another preferred embodiment, the compound of formula (VIIIb) is selected from the group consisting of

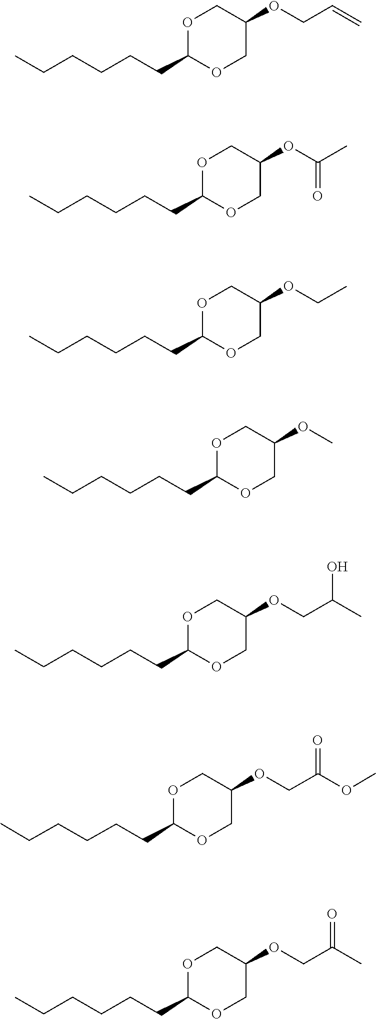

Another aspect of the present invention relates to a mixture comprising one or more compound(s) of formula (I) and one or more compound(s) of formula (VIII)

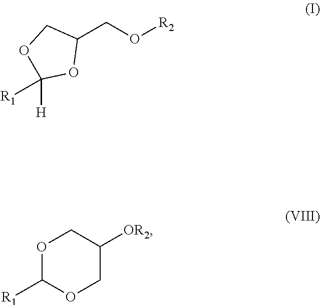

wherein
R₁ is selected from the group consisting of n-pentyl, n-hexyl, and n-heptyl, preferably is selected either n-hexyl or n-heptyl,
and
R₂ is selected from the group consisting of H, methyl, ethyl, n-propyl, isopropyl, allyl,

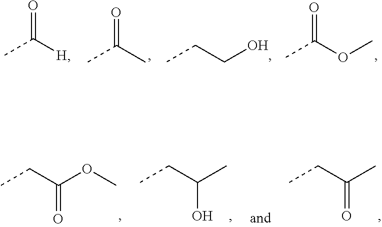

preferably is selected from the group consisting of methyl, ethyl, n-propyl, allyl,

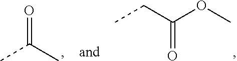

most preferably is allyl.

Another aspect of the present invention relates to the use of a compound of formula (I) and/or of a compound of formula (VIII)

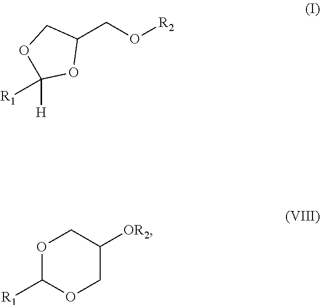

wherein R₁ is selected from the group consisting of n-pentyl, n-hexyl, and n-heptyl, preferably is either n-hexyl or n-heptyl,
and
R₂ is selected from the group consisting of H, methyl, ethyl, n-propyl, isopropyl, allyl,

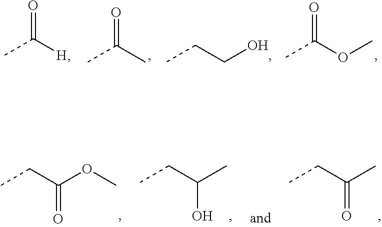

preferably is selected from the group consisting of methyl, ethyl, n-propyl, allyl,

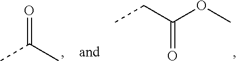

most preferably is allyl,
as fragrance and/or aroma substance(s), preferably
(i) with a transparent soft white-floral jasminic note
and/or
(ii) to impart, modify, and/or enhance one or more odor and/or taste notes selected from the group consisting of jasmine, lily of the valley, orris, violet, rose, ylang ylang, apple, pear, banana, pineapple, plum, cedarwood, sandalwood, vetiver, and musk
and/or
(iii) to impart, modify, and/or enhance, preferably to impart and/or enhance, one or more odor and/or taste effects selected from the group consisting of creaminess, floral freshness, radiance, and luminosity.

According to a preferred embodiment, the compound of formula (I) used according to the invention comprises at least 10, 20, 30, 40, 50, 60, 70, 80, or 90 wt. %, preferably more than 50 wt. %, of biobased carbon (i.e. non-fossil fuel based carbon), based on the total weight of the molecule. More preferably, the compound of formula (I) used according to the invention only contains biobased carbon.

According to another preferred embodiment of the present invention, more than 50% of the carbon atoms contained in the compound of formula (I) used according to the invention are biobased, based on the total number of carbon atoms contained in the compound of formula (I) used according to the invention. More preferably, more than 60%, 70%, 75%, 80%, 85%, or 90% of the carbon atoms contained in the compound of formula (I) used according to the invention are biobased, based on the total number of carbon atoms contained in the compound of formula (I) used according to the invention. Most preferably, more than 75% of the carbon atoms contained in the compound of formula (I) used according to the invention are biobased, based on the total number of carbon atoms contained in the compound of formula (I) used according to the invention.

According to a preferred embodiment, the compound of formula (VIII) used according to the invention comprises at least 10, 20, 30, 40, 50, 60, 70, 80, or 90 wt. %, preferably more than 50 wt. %, of biobased carbon (i.e. non-fossil fuel based carbon), based on the total weight of the molecule. More preferably, the compound of formula (VIII) used according to the invention only contains biobased carbon.

According to another preferred embodiment of the present invention, more than 50% of the carbon atoms contained in the compound of formula (VIII) used according to the invention are biobased, based on the total number of carbon atoms contained in the compound of formula (VIII) used according to the invention. More preferably, more than 60%, 70%, 75%, 80%, 85%, or 90% of the carbon atoms contained in the compound of formula (VIII) used according to the invention are biobased, based on the total number of carbon atoms contained in the compound of formula (VIII) used according to the invention. Most preferably, more than 75% of the carbon atoms contained in the compound of formula (VIII) used according to the invention are biobased, based on the total number of carbon atoms contained in the compound of formula (VIII) used according to the invention.

A preferred embodiment of the use according to the invention relates to the use of a compound of formula (I)

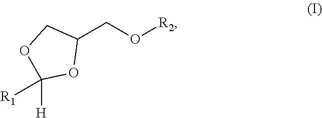

wherein R₁ is selected from the group consisting of n-pentyl, n-hexyl, and n-heptyl, preferably is either n-hexyl or n-heptyl,
and
R₂ is selected from the group consisting of H, methyl, ethyl, n-propyl, isopropyl, allyl,

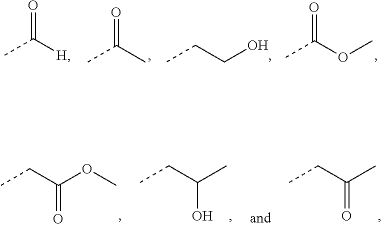

preferably is selected from the group consisting of methyl, ethyl, n-propyl, allyl,

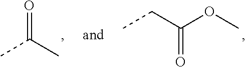

most preferably is allyl,
as fragrance and/or aroma substance(s), preferably
(i) with a transparent soft white-floral jasminic note
and/or
(ii) to impart, modify, and/or enhance one or more odor and/or taste notes selected from the group consisting of jasmine, lily of the valley, orris, violet, rose, ylang ylang, apple, pear, banana, pineapple, plum, cedarwood, sandalwood, vetiver, and musk and/or
(iii) to impart, modify, and/or enhance, preferably to impart and/or enhance, one or more odor and/or taste effects selected from the group consisting of creaminess, floral freshness, radiance, and luminosity.

A preferred embodiment of the use according to the invention relates to the use of a compound of formula (VIII)

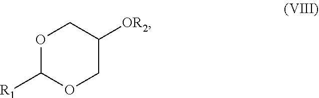

wherein R₁ is selected from the group consisting of n-pentyl, n-hexyl, and n-heptyl, preferably is either n-hexyl or n-heptyl,
and
R₂ is selected from the group consisting of H, methyl, ethyl, n-propyl, isopropyl, allyl,

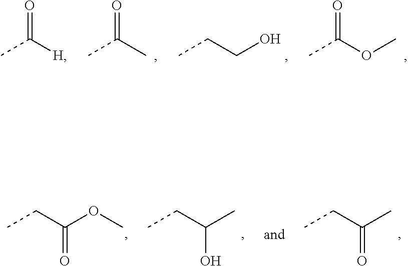

preferably is selected from the group consisting of methyl, ethyl, n-propyl, allyl,

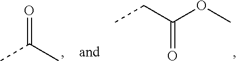

more preferably is allyl,
as fragrance and/or aroma substance(s), preferably
(i) with a transparent soft white-floral jasminic note
and/or
(ii) to impart, modify, and/or enhance one or more odor and/or taste notes selected from the group consisting of jasmine, lily of the valley, orris, violet, rose, ylang ylang, apple, pear, banana, pineapple, plum, cedarwood, sandalwood, vetiver, and musk
and/or
(iii) to impart, modify, and/or enhance, preferably to impart and/or enhance, one or more odor and/or taste effects selected from the group consisting of creaminess, floral freshness, radiance, and luminosity.

A preferred embodiment of the use according to the invention relates to the use of one or more compound(s) selected from the group consisting of

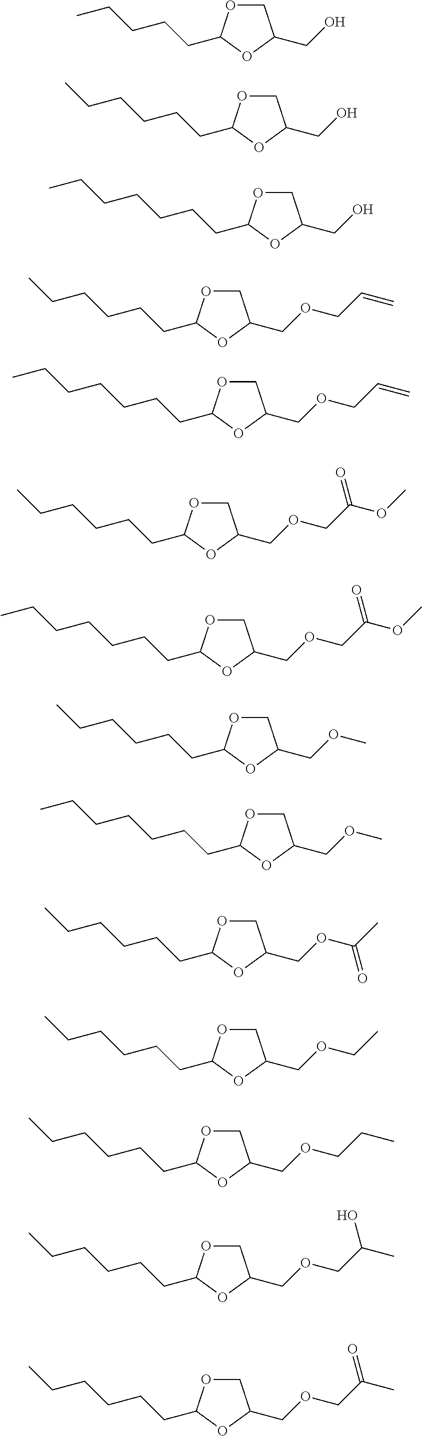

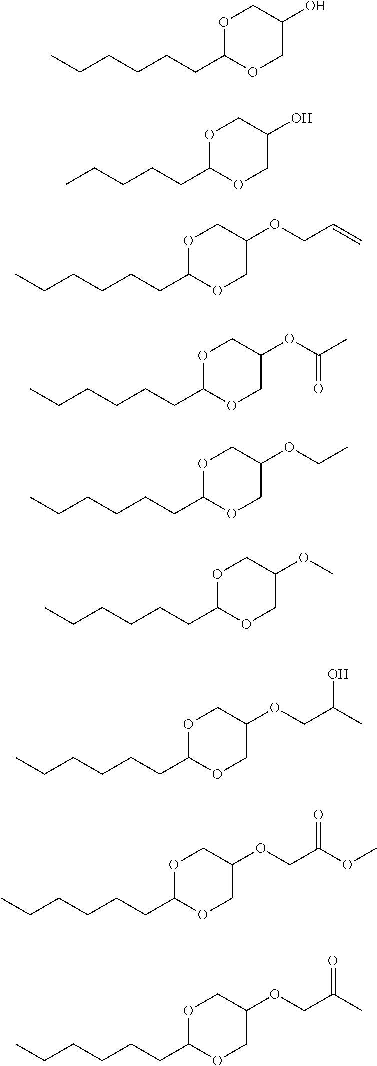

as fragrance and/or aroma substance(s), preferably
(i) with a transparent soft white-floral jasminic note
and/or
(ii) to impart, modify, and/or enhance one or more odor and/or taste notes selected from the group consisting of jasmine, lily of the valley, orris, violet, rose, ylang ylang, apple, pear, banana, pineapple, plum, cedarwood, sandalwood, vetiver, and musk and/or
(iii) to impart, modify, and/or enhance, preferably to impart and/or enhance, one or more odor and/or taste effects selected from the group consisting of creaminess, floral freshness, radiance, and luminosity.

Another preferred embodiment of the use according to the invention relates to the use of one or more compound(s) selected from the group consisting of

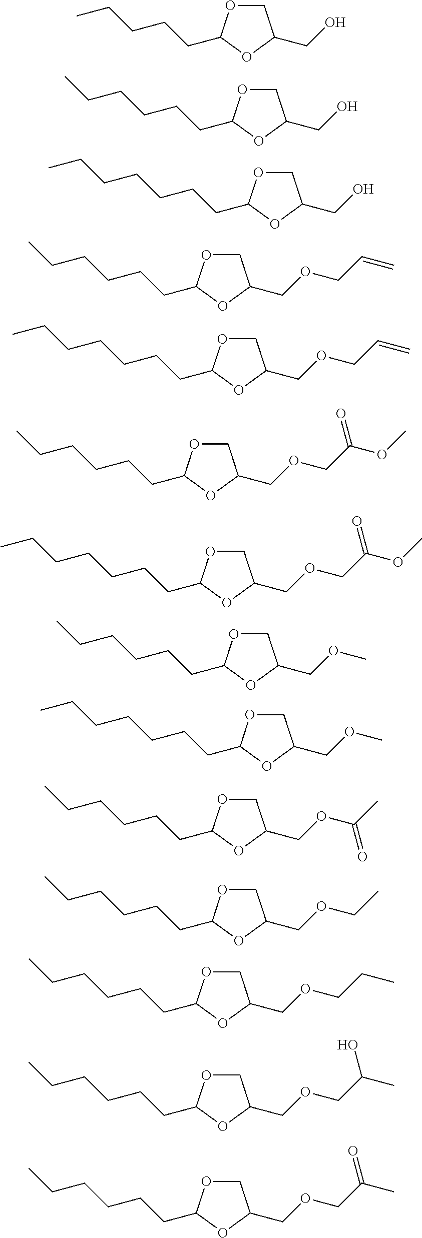

as fragrance and/or aroma substance(s), preferably
(i) with a transparent soft white-floral jasminic note
and/or
(ii) to impart, modify, and/or enhance one or more odor and/or taste notes selected from the group consisting of jasmine, lily of the valley, orris, violet, rose, ylang ylang, apple, pear, banana, pineapple, plum, cedarwood, sandalwood, vetiver, and musk
and/or
(iii) to impart, modify, and/or enhance, preferably to impart and/or enhance, one or more odor and/or taste effects selected from the group consisting of creaminess, floral freshness, radiance, and luminosity.

Another preferred embodiment of the use according to the invention relates to the use of one or more compound(s) selected from the group consisting of

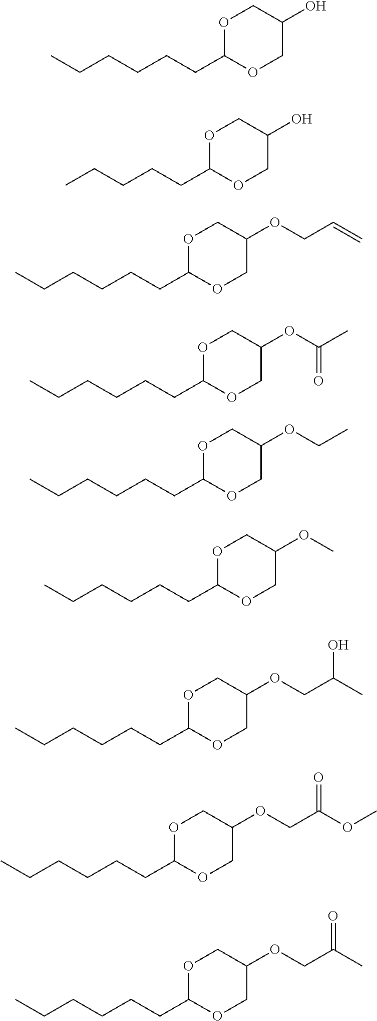

as fragrance and/or aroma substance(s), preferably
(i) with a transparent soft white-floral jasminic note
and/or
(ii) to impart, modify, and/or enhance one or more odor and/or taste notes selected from the group consisting of jasmine, lily of the valley, orris, violet, rose, ylang ylang, apple, pear, banana, pineapple, plum, cedarwood, sandalwood, vetiver, and musk
and/or
(iii) to impart, modify, and/or enhance, preferably to impart and/or enhance, one or more odor and/or taste effects selected from the group consisting of creaminess, floral freshness, radiance, and luminosity.

Another preferred embodiment of the use according to the invention relates to the use of a compound of formula (I) and/or of a compound of formula (VIII) as defined herein as a solvent and/or filler, preferably as a solvent and/or filler and (at the same time) as a fragrance and/or aroma substance with a transparent soft white-floral jasminic note.

Another preferred embodiment of the use according to the invention relates to the use of a compound of formula (I) as defined herein as a solvent and/or filler, preferably as a solvent and/or filler and as a fragrance and/or aroma substance with a transparent soft white-floral jasminic note or as a fragrance and/or aroma substance for imparting, modifying, and/or enhancing one or more odor and/or taste notes selected from the group consisting of jasmine, lily of the valley, orris, violet, rose, ylang ylang, apple, pear, banana, pineapple, plum, cedarwood, sandalwood, vetiver, and musk or as a fragrance and/or aroma substance for imparting, modifying, and/or enhancing, preferably for imparting and/or enhancing, one or more odor and/or taste effects selected from the group consisting of creaminess, floral freshness, radiance, and luminosity.

Another preferred embodiment of the use according to the invention relates to the use of a compound of formula (I) as defined herein as a solvent.

Another preferred embodiment of the use according to the invention relates to the use of a compound of formula (I) as defined herein as a filler.

Another preferred embodiment of the use according to the invention relates to the use of a compound of formula (I) as defined herein as a solvent and as a fragrance and/or aroma substance with a transparent soft white-floral jasminic note.

Another preferred embodiment of the use according to the invention relates to the use of a compound of formula (I) as defined herein as a filler and as a fragrance and/or aroma substance with a transparent soft white-floral jasminic note.

Another preferred embodiment of the use according to the invention relates to the use of a compound of formula (VIII) as defined herein as a solvent and/or filler, preferably as a solvent and/or filler and as a fragrance and/or aroma substance with a transparent soft white-floral jasminic note or as a fragrance and/or aroma substance for imparting, modifying, and/or enhancing one or more odor and/or taste notes selected from the group consisting of jasmine, lily of the valley, orris, violet, rose, ylang ylang, apple, pear, banana, pineapple, plum, cedarwood, sandalwood, vetiver, and musk or as a fragrance and/or aroma substance for imparting, modifying, and/or enhancing, preferably for imparting and/or enhancing, one or more odor and/or taste effects selected from the group consisting of creaminess, floral freshness, radiance, and luminosity.

Another preferred embodiment of the use according to the invention relates to the use of a compound of formula (VIII) as defined herein as a solvent.

Another preferred embodiment of the use according to the invention relates to the use of a compound of formula (VIII) as defined herein as a filler.

Another preferred embodiment of the use according to the invention relates to the use of a compound of formula (VIII) as defined herein as a solvent and as a fragrance and/or aroma substance with a transparent soft white-floral jasminic note.

Another preferred embodiment of the use according to the invention relates to the use of a compound of formula (VIII) as defined herein as a filler and as a fragrance and/or aroma substance with a transparent soft white-floral jasminic note.

Another aspect of the present invention relates to a fragrance and/or aroma composition, preferably perfume oil, comprising one or more compound(s) of formula (I) according to the invention and/or a mixture according to the invention and/or one or more compound(s) of formula (VIII) according to the invention and/or one or more compound(s) of formula (I) as defined herein and/or one or more compound(s) of formula (VIII) as defined herein, and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

A preferred embodiment relates to a fragrance and/or aroma composition, preferably perfume oil, comprising one or more compound(s) of formula (I) according to the invention, and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

Another preferred embodiment relates to a fragrance and/or aroma composition, preferably perfume oil, comprising a mixture according to the invention,

and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

Another preferred embodiment relates to a fragrance and/or aroma composition, preferably perfume oil, comprising one or more compound(s) of formula (VIII) according to the invention,

and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

Another preferred embodiment relates to a fragrance and/or aroma composition, preferably perfume oil, comprising one or more compound(s) of formula (I) as defined herein,

and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

Another preferred embodiment relates to a fragrance and/or aroma composition, preferably perfume oil, comprising one or more compound(s) of formula (VIII) as defined herein,

and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

Another preferred embodiment relates to a fragrance and/or aroma composition, preferably perfume oil, comprising one or more compound(s) of formula (I) according to the invention and/or a mixture according to the invention and/or one or more compound(s) of formula (I) as defined herein,

and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

Another preferred embodiment relates to a fragrance and/or aroma composition, preferably perfume oil, comprising one or more compound(s) of formula (VIII) according to the invention and/or a mixture according to the invention and/or one or more compound(s) of formula (VIII) as defined herein,

and preferably further comprising one or more additional fragrance and/or aroma substances selected from the group consisting of Agrumex (2-tert-butylcyclohexyl acetate), Ethylmethyl Butyrate-2 (ethyl 2-methylbutanoate), Isoamyl Acetate (3-methylbutan-1-ol), Dimethyl Benzyl Carbinyl Butyrate (1,1-dimethyl-2-phenylethyl butyrate), Hexyl Acetate (hexyl acetate), Allyl Cyclohexyl Propionate (prop-2-en-1-yl 3-cyclohexylpropanoate), Dihydro Myrcenol (2,6-dimethyloct-7-en-2-ol), Ethyl Linalool (3,7-dimethylnona-1,6-dien-3-ol), Helional (3-(1′,3′-benzodioxol-5′-yl)-2-methylpropanal), Cyclamen Aldehyde (2-methyl-3-[4′-(propan-2′-yl)phenyl]propanal), Jasmon Cis (3-methyl-2-pent-2-enylcyclopent-2-enone), Benzyl Acetate (benzyl acetate), methyl dihydrojasmonate (methyl 2-(3′-oxo-2′-pentylcyclopentyl)acetate), Methyl Anthranilate (methyl 2-aminobenzoate), Methyl Ionone 70 ((1E)-1-(2,6,6-trimethylcyclohex-2-en-1-yl)pent-1-en-3-one; (3E)-3-methyl-4-(2′,6′,6′-trimethylcyclohex-2′-en-1-yl)but-3-en-2-one), Ionone Beta (4-(2′,6′,6′-trimethylcyclohex-1′-en-1′-yl)but-3-en-2-one), Geraniol (3,7-dimethylocta-2,6-dien-1-ol, Damascenone (1-(2′,6′,6′-trimethylcyclohexa-1′,3′-dien-1′-yl)but-2-en-1-one)), Phenylethyl Alcohol (2-phenylethanol), Benzyl Salicylate (benzyl salicylate), Hexyl Salicylate (hexyl salicylate), Ambroxide ((3aR,5aS,9aS,9bR)-3a,6,6,9a-tetramethyldodecahydronaphtho[2,1-b]furan), Iso E Super (reaction mass from the acidic cyclization of 1-(1′,6′-dimethyl-4′-(4″-methylpent-3″-en-1″-yl)cyclohex-3′-en-1′-yl)ethan-1-one), Cedryl Acetate (reaction mass from the esterification of cedrol rich cedarwood oil fractions), Sandranol® (2-ethyl-4-(2′,2′,3′-trimethylcyclopent-3′-en-1′-yl)but-2′-en-1′-ol), Polysantol ((4E)-3,3-dimethyl-5-(2′,2′,3′-trimethylcyclopent-3′-en-1′-yl)pent-4-en-2-ol), Vetiveryl Acetate (reaction mass from the acetylation of vetiver oil), Ambrettolide (oxacycloheptadec-10-en-2-one), Ethylene Brassylate (1,4-dioxacycloheptadecane-5,17-dione), Globalide ((12E)-1-oxacyclohexadec-12-en-2-one; (12Z)-1-oxacyclohexadec-12-en-2-one; (13E)-1-oxacyclohexadec-13-en-2-one; (13Z)-1-oxacyclohexadec-13-en-2-one)), and Muscenone (3-Methyl-5-cyclopentadecen-1-one).

Another preferred embodiment relates to a fragrance and/or aroma composition, wherein the total amount of the one or more compound(s) of formula (I), if present, and of the one or more compounds of formula (VIII), if present, contained in the composition is sufficient
(a) to mask and/or to reduce an unpleasant odor impression of at least one additional fragrance and/or aroma substance in the fragrance and/or aroma composition,
and/or
(b) to enhance a pleasant odor impression of at least one additional fragrance and/or aroma substance in the fragrance and/or aroma composition.

Another preferred embodiment relates to a fragrance and/or aroma composition, wherein the total amount of the one or more compound(s) of formula (I) contained in the composition is sufficient
(a) to mask and/or to reduce an unpleasant odor impression of at least one additional fragrance and/or aroma substance in the fragrance and/or aroma composition,
and/or
(b) to enhance a pleasant odor impression of at least one additional fragrance and/or aroma substance in the fragrance and/or aroma composition.

Another preferred embodiment relates to a fragrance and/or aroma composition, wherein the total amount of the one or more compound(s) of formula (VIII) contained in the composition is sufficient
(a) to mask and/or to reduce an unpleasant odor impression of at least one additional fragrance and/or aroma substance in the fragrance and/or aroma composition,
and/or
(b) to enhance a pleasant odor impression of at least one additional fragrance and/or aroma substance in the fragrance and/or aroma composition.

Another aspect of the present invention relates to a product comprising one or more compound(s) of formula (I) according to the invention and/or a mixture according to the invention and/or one or more compound(s) of formula (VIII) according to the invention and/or a fragrance and/or aroma composition according to the invention and/or one or more compound(s) of formula (I) as defined herein and/or one or more compound(s) of formula (VIII) as defined herein,
preferably in a sensorially effective amount,
more preferably wherein the total amount of the one or more compound(s) of formula (I) as defined herein, if present, and of the one or more compound(s) of formula (VIII) as defined herein, if present, contained in the product is in a range of from 0.1 to 65 wt. %, preferably from 1 to 40 wt. %, more preferably from 10 to 20 wt. %, based on the total weight of the product,
most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

A preferred embodiment of the product according to the invention relates to a product comprising one or more compound(s) of formula (I) according to the invention,
preferably in a sensorially effective amount,
more preferably wherein the total amount of the one or more compound(s) of formula (I) according to the invention contained in the product is in a range of from 0.1 to 65 wt. %, preferably from 1 to 40 wt. %, more preferably from 10 to 20 wt. %, based on the total weight of the product,
most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another preferred embodiment of the product according to the invention relates to a product comprising a mixture according to the invention,
preferably in a sensorially effective amount,
more preferably wherein the total amount of the one or more compound(s) of formula (I) as defined herein, if present, and of the one or more compound(s) of formula (VIII) as defined herein, if present, contained in the product is in a range of from 0.1 to 65 wt. %, preferably from 1 to 40 wt. %, more preferably from 10 to 20 wt. %, based on the total weight of the product,
most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another preferred embodiment of the product according to the invention relates to a product comprising one or more compound(s) of formula (VIII) according to the invention,
preferably in a sensorially effective amount,
more preferably wherein the total amount of the one or more compound(s) of formula (VIII) according to the invention contained in the product is in a range of from 0.1 to 65 wt. %, preferably from 1 to 40 wt. %, more preferably from 10 to 20 wt. %, based on the total weight of the product, most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another preferred embodiment of the product according to the invention relates to a product comprising a fragrance and/or aroma composition according to the invention,
preferably in a sensorially effective amount,
more preferably wherein the total amount of the one or more compound(s) of formula (I) as defined herein, if present, and of the one or more compound(s) of formula (VIII) as defined herein, if present, contained in the product is in a range of from 0.1 to 65 wt. %, preferably from 1 to 40 wt. %, more preferably from 10 to 20 wt. %, based on the total weight of the product,
most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another preferred embodiment of the product according to the invention relates to a product comprising one or more compound(s) of formula (I) as defined herein,
preferably in a sensorially effective amount,
more preferably wherein the total amount of the one or more compound(s) of formula (I) as defined herein contained in the product is in a range of from 0.1 to 65 wt. %, preferably from
1 to 40 wt. %, more preferably from 10 to 20 wt. %, based on the total weight of the product, most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another preferred embodiment of the product according to the invention relates to a product comprising one or more compound(s) of formula (VIII) as defined herein,
preferably in a sensorially effective amount,
more preferably wherein the total amount of the one or more compound(s) of formula (VIII) as defined herein contained in the product is in a range of from 0.1 to 65 wt. %, preferably from 1 to 40 wt. %, more preferably from 10 to 20 wt. %, based on the total weight of the product,
most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another preferred embodiment of the product according to the invention relates to a product comprising one or more compound(s) of formula (I) according to the invention and/or a mixture according to the invention and/or a fragrance and/or aroma composition according to the invention and/or one or more compound(s) of formula (I) as defined herein,
preferably in a sensorially effective amount,
more preferably wherein the total amount of the one or more compound(s) of formula (I) as defined herein contained in the product is in a range of from 0.1 to 65 wt. %, preferably from 1 to 40 wt. %, more preferably from 10 to 20 wt. %, based on the total weight of the product,
most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another preferred embodiment of the product according to the invention relates to a product comprising one or more compound(s) of formula (VIII) according to the invention and/or a mixture according to the invention and/or a fragrance and/or aroma composition according to the invention and/or one or more compound(s) of formula (VIII) as defined herein,
preferably in a sensorially effective amount,
more preferably wherein the total amount of the one or more compound(s) of formula (VIII) as defined herein contained in the product is in a range of from 0.1 to 65 wt. %, preferably from 1 to 40 wt. %, more preferably from 10 to 20 wt. %, based on the total weight of the product,
most preferably wherein product is selected from the group consisting of perfume extraits, eau de parfums, eau de toilettes, eau de colognes, pro perfume delivery systems, splash colognes, shaving creams, soaps and lotions, pre-shave products, after-shave products, preferably creams and lotions, body care products, preferably shower gels, oils, foams and soaps, body scrubs, body lotions, body tan products, body spray, baby-care products, hand creams and lotions, foot creams, tonics, and lotions, depilatory creams, tonics, and lotions, tanning creams, sprays, and lotions, facial beauty care products, cosmetic emulsions, skin tonics, skin brightening products, sun protection products and after sun products, hair care products, preferably shampooing and conditioning products, products for hair styling such as hair gels, hair waxes, hair mousses, and hair sprays, hair pigmentation products, decorative cosmetic products, make-up removing products, nail care products, nail polish removing products, deodorants, antiperspirants, candles, lamp oils, incense sticks, insecticides, repellents and fuels, air freshening products, incense sticks, anti-malodor products, home care products, dishwashing products, car cleaning products, furniture care products, aromatherapy products, perfumed refreshing tissues, facial tissues, paper handkerchiefs, dryer sheets, wet toilet paper, acidic, alkaline and neutral detergents, textile fresheners, ironing aids, liquid detergents, powdery detergents, laundry sheets, foams, and pods, laundry pre-treatments, fabric softeners, laundry soaps, bleaching agents, dipping agents, stain removal agents, washing tablets, disinfectants, surface disinfectants, air fresheners, aerosol sprays, waxes, and polishes.

Another aspect of the present invention relates to a method for manufacturing a product according to the invention, comprising or consisting of the following steps:
(i) Providing one or more compound(s) of formula (I) according to the invention and/or a mixture according to the invention and/or one or more compound(s) of formula (VIII) according to the invention and/or a fragrance and/or aroma composition according to the invention and/or one or more compound(s) of formula (I) as defined herein and/or one or more compound(s) of formula (VIII) as herein,
(ii) providing the further ingredients of the product,
(iii) mixing the further ingredients of the product provided in step (ii) with, preferably a sensorially effective amount of, the compound(s) and/or mixture and/or composition provided in step (i).

Preferably, the method for manufacturing a product according to the invention comprises or consists of the following steps:
(i) Providing one or more compound(s) of formula (I) according to the invention,
(ii) providing the further ingredients of the product,
(iii) mixing the further ingredients of the product provided in step (ii) with, preferably a sensorially effective amount of, the compound(s) provided in step (i).

Preferably, the method for manufacturing a product according to the invention comprises or consists of the following steps:
(i) Providing a mixture according to the invention,
(ii) providing the further ingredients of the product,
(iii) mixing the further ingredients of the product provided in step (ii) with, preferably a sensorially effective amount of, the mixture provided in step (i).

Preferably, the method for manufacturing a product according to the invention comprises or consists of the following steps:
(i) Providing one or more compound(s) of formula (VIII) according to the invention,
(ii) providing the further ingredients of the product,
(iii) mixing the further ingredients of the product provided in step (ii) with, preferably a sensorially effective amount of, the compound(s) provided in step (i).

Preferably, the method for manufacturing a product according to the invention comprises or consists of the following steps:
(i) Providing a fragrance and/or aroma composition according to the invention,
(ii) providing the further ingredients of the product,
(iii) mixing the further ingredients of the product provided in step (ii) with, preferably a sensorially effective amount of, the composition provided in step (i).

Preferably, the method for manufacturing a product according to the invention comprises or consists of the following steps:
(i) Providing one or more compound(s) of formula (I) as defined herein,
(ii) providing the further ingredients of the product,
(iii) mixing the further ingredients of the product provided in step (ii) with, preferably a sensorially effective amount of, the compound(s) provided in step (i).

Preferably, the method for manufacturing a product according to the invention comprises or consists of the following steps:
(i) Providing one or more compound(s) of formula (VIII) as herein,
(ii) providing the further ingredients of the product,
(iii) mixing the further ingredients of the product provided in step (ii) with, preferably a sensorially effective amount of, the compound(s) provided in step (i).

Another aspect of the present invention relates to a method for providing and/or enhancing a transparent soft white-floral jasminic note to/in a product, preferably a product as defined herein, comprising or consisting of the following steps:
(a) Identifying a product to/in which a transparent soft white-floral jasminic note is to be provided and/or enhanced, preferably a product as defined herein, and
(b) incorporating one or more compound(s) of formula (I) according to the invention and/or a mixture according to the invention and/or one or more compound(s) of formula (VIII) according to the invention and/or one or more compound(s) of formula (I) as defined herein and/or one or more compound(s) of formula (VIII) as defined herein and/or a fragrance and/or aroma composition according to the invention into the product in an amount sufficient to provide and/or enhance a/the transparent soft white-floral jasminic note to/in the product.

Preferably, the method for providing and/or enhancing a transparent soft white-floral jasminic note to/in a product, preferably a product as defined herein, comprises or consists of the following steps:
(a) Identifying a product to/in which a transparent soft white-floral jasminic note is to be provided and/or enhanced, preferably a product as defined herein, and
(b) incorporating one or more compound(s) of formula (I) according to the invention or as defined herein into the product in an amount sufficient to provide and/or enhance a/the transparent soft white-floral jasminic note to/in the product.

Preferably, the method for providing and/or enhancing a transparent soft white-floral jasminic note to/in a product, preferably a product as defined herein, comprises or consists of the following steps:
(a) Identifying a product to/in which a transparent soft white-floral jasminic note is to be provided and/or enhanced, preferably a product as defined herein, and
(b) incorporating a mixture according to the invention into the product in an amount sufficient to provide and/or enhance a/the transparent soft white-floral jasminic note to/in the product.

Preferably, the method for providing and/or enhancing a transparent soft white-floral jasminic note to/in a product, preferably a product as defined herein, comprises or consists of the following steps:
(a) Identifying a product to/in which a transparent soft white-floral jasminic note is to be provided and/or enhanced, preferably a product as defined herein, and
(b) incorporating one or more compound(s) of formula (VIII) according to the invention or as defined herein into the product in an amount sufficient to provide and/or enhance a/the transparent soft white-floral jasminic note to/in the product.

Preferably, the method for providing and/or enhancing a transparent soft white-floral jasminic note to/in a product, preferably a product as defined herein, comprises or consists of the following steps:
(a) Identifying a product to/in which a transparent soft white-floral jasminic note is to be provided and/or enhanced, preferably a product as defined herein, and
(b) incorporating a fragrance and/or aroma composition according to the invention into the product in an amount sufficient to provide and/or enhance a/the transparent soft white-floral jasminic note to/in the product.

Another aspect of the present invention relates to the use of
one or more compound(s) of formula (I) according to the invention or as defined herein

and/or
one or more compound(s) of formula (VIII) according to the invention or as defined herein

and/or
a mixture as defined herein

and/or
a fragrance and/or aroma composition as defined herein

and/or
a product as defined herein

to perfume textiles, hair, skin, surfaces and/or ambient air, preferably with a transparent soft white-floral jasminic note.

A preferred embodiment relates to the use of one or more compound(s) of formula (I) according to the invention or as defined herein to perfume textiles, hair, skin, surfaces and/or ambient air, preferably with a transparent soft white-floral jasminic note.

A preferred embodiment relates to the use of one or more compound(s) of formula (VIII) according to the invention or as defined herein to perfume textiles, hair, skin, surfaces and/or ambient air, preferably with a transparent soft white-floral jasminic note. (Preferred) embodiments of the compounds according to the invention correspond to or can be derived from the (preferred) embodiments of the mixtures according to the invention which are explained above or vice versa. (Preferred) embodiments of the compounds or mixtures according to the invention correspond to or can be derived from the (preferred) embodiments of the methods according to the invention which are explained above or vice versa. (Preferred) embodiments of the compounds or mixtures according to the invention correspond to or can be derived from the (preferred) embodiments of the uses according to the invention which are explained above or vice versa. (Preferred) embodiments of the compounds or mixtures according to the invention correspond to or can be derived from the (preferred) embodiments of the fragrance and/or aroma compositions according to the invention which are explained above or vice versa. (Preferred) embodiments of the compounds or mixtures according to the invention correspond to or can be derived from the (preferred) embodiments of the products according to the invention which are explained above or vice versa. (Preferred) embodiments of the methods according to the invention correspond to or can be derived from the (preferred) embodiments of the uses according to the invention which are explained above or vice versa. Moreover, the (preferred) embodiments described herein can be arbitrarily combined with each other as long as technically sensible.

The invention will now be described in more detail hereinafter with references to the examples. Further aspects of the present invention are disclosed in the accompanying claims. Unless otherwise stated, all values refer to weight-% (wt. %).

### EXAMPLES

### Example 1

### Preparation of 4-(allyloxymethyl)-2-hexyl-1,3-dioxolane

pTsOH (7.61 g, 39.4 mmol) was added in portions to a stirred solution of petrochemical heptanal (232 g, 1.97 mol, supplied by Thermo Scientific) and 3-allyloxy-1,2-propanediol (263 g, 1.97 mol) in cyclohexane (1.0 l). The reaction mixture was then heated to reflux in a water separator for 2 h, and subsequently allowed to cool to room temperature. After washing with brine (300 ml), saturated aq. NaHCO₃ (2×150 ml) and brine (150 ml) again, the crude material was dried (Na₂SO₄) and concentrated under reduced pressure. Distillation of the resulting residue (443 g) provided 4-(allyloxymethyl)-2-hexyl-1,3-dioxolane (364 g, 99% purity, 80% yield) as a colorless odoriferous liquid mixture of cis:trans isomers (55:45).

¹H NMR (400 MHz, CDCl₃) δ 5.90 (ddt, J=17.5, 11.0, 6.0 Hz, 2H), 5.28 (dq, J=17.5, 2.0 Hz, 2H), 5.19 (dq, J=10.5, 1.5 Hz, 2H), 4.98 (t, J=5.0 Hz, 1H), 4.89 (t, J=5.0 Hz, 1H), 4.31-4.18 (m, 2H), 4.13 (dd, J=8.5, 6.6 Hz, 1H), 4.09-3.98 (m, 4H), 3.91 (dd, J=8.0, 7.0 Hz, 1H), 3.77 (dd, J=8.0, 5.0 Hz, 1H), 3.64 (dd, J=8.5, 7.0 Hz, 1H), 3.54 (td, J=10.0, 6.0 Hz, 2H), 3.44 (ddd, J=19.0, 10.0, 5.5 Hz, 2H), 1.71-1.59 (m, 4H), 1.47-1.37 (m, 4H), 1.37-1.20 (m, 12H), 0.87 (t, 6H).

¹³C NMR (101 MHz, CDCl₃) δ 134.5, 117.4, 117.3, 105.2, 104.7, 74.8, 74.5, 72.5, 71.1, 70.5, 67.5, 67.5, 34.0, 34.0, 31.8, 29.2, 23.9, 23.9, 22.6, 14.1 ppm.

GC-MS (70 eV), trans-isomer: m/z (%)=144 (8), 143 (100), 97 (36), 69 (5), 57 (10), 55 (26), 43 (8), 41 (36), 39 (5), 29 (5).

GC-MS (70 eV), cis-isomer: m/z (%)=144 (8), 143 (100), 97 (32), 69 (5), 57 (11), 55 (27), 43 (8), 41 (36), 39 (5), 29 (5).

Odor description of the trans-isomer: Fresh, citric, jasminic, methyl dihydrojasmonate. Odor description of the cis-isomer: Soft, floral, jasminic, slightly greasy.

Odor description of the cis:trans mixture (55:45): Floral, soft, jasminic, reminiscent of methyl dihydrojasmonate, slightly oily.

### Example 2

### 2.1 Preparation of 4-(allyloxymethyl)-2-hexyl-1,3-dioxolane, 54% biobased

pTsOH (7.23 g, 37.5 mmol) was added in portions to a stirred solution of biobased heptanal (220 g, 1.87 mol, supplied by Arkema Deutschland) and 3-allyloxy-1,2-propanediol (250 g, 1.87 mol) in cyclohexane (400 ml). The reaction mixture was then heated to reflux in a water separator for 2 h, and subsequently allowed to cool to room temperature. After washing with brine (200 ml), saturated aq. NaHCO₃ (200 ml) and brine (2×150 ml) again, the crude material was dried (Na₂SO₄) and concentrated under reduced pressure. Distillation of the resulting residue (423 g) provide 54% biobased 4-(allyloxymethyl)-2-hexyl-1,3-dioxolane (287 g, 99% purity, 67% yield) as a colorless odoriferous liquid mixture of cis:trans isomers (61:38).

¹H NMR (400 MHz, CDCl₃) δ 5.90 (ddt, J=17.5, 11.0, 6.0 Hz, 2H), 5.28 (dq, J=17.5, 2.0 Hz, 2H), 5.19 (dq, J=10.5, 1.5 Hz, 2H), 4.98 (t, J=5.0 Hz, 1H), 4.89 (t, J=5.0 Hz, 1H), 4.31-4.18 (m, 2H), 4.13 (dd, J=8.5, 6.6 Hz, 1H), 4.09-3.98 (m, 4H), 3.91 (dd, J=8.0, 7.0 Hz, 1H), 3.77 (dd, J=8.0, 5.0 Hz, 1H), 3.64 (dd, J=8.5, 7.0 Hz, 1H), 3.54 (td, J=10.0, 6.0 Hz, 2H), 3.44 (ddd, J=19.0, 10.0, 5.5 Hz, 2H), 1.71-1.59 (m, 4H), 1.47-1.37 (m, 4H), 1.37-1.20 (m, 12H), 0.87 (t, 6H). ¹³C NMR (101 MHz, CDCl₃) δ 134.5, 117.4, 117.3, 105.2, 104.7, 74.8, 74.5, 72.5, 71.1, 70.5, 67.5, 67.5, 34.0, 34.0, 31.8, 29.2, 23.9, 23.9, 22.6, 14.1 ppm.

GC-MS (70 eV), trans-isomer: m/z (%)=144 (7), 143 (100), 97 (50), 71 (6), 69 (8), 57 (16), 55 (51), 43 (16), 42 (5), 41 (79), 39 (8), 29 (13).

GC-MS (70 eV), cis-isomer: m/z (%)=144 (8), 143 (100), 97 (54), 71 (7), 69 (10), 57 (18), 55 (57), 43 (16), 42 (5), 41 (81), 39 (8), 29 (14).

Odor description of the cis:trans mixture (61:38): Floral, soft-jasminic airy, solar, reminiscent of methyl dihydrojasmonate, faint champignon note.

### 2.2 Separation of the cis- and trans-isomers of 4-(allyloxymethyl)-2-hexyl-1,3-dioxolane

The 4-(allyloxymethyl)-2-hexyl-1,3-dioxolane (45.2 g, 99% purity), obtained above in Example 2.1 with a cis:trans ratio of 61:38, was subjected to a distillation in a Spaltrohr-column system at 1-3 mbar. Thus, the following fractions containing the enriched or pure cis-configured 4-(allyloxymethyl)-2-hexyl-1,3-dioxolane were obtained:
(0.20 g, 65% purity) swamp temperature 105° C., head temperature 86° C.,
(0.30 g, 72% purity) swamp temperature 105° C., head temperature 88° C.,
(0.70 g, 85% purity) swamp temperature 105° C., head temperature 90° C.,
(1.30 g, 94% purity) swamp temperature 106° C., head temperature 91° C.,
(0.90 g, 98% purity) swamp temperature 109° C., head temperature 92° C.,
(2.40 g, 99% purity) swamp temperature 109° C., head temperature 92° C.,
(1.40 g, 99% purity) swamp temperature 109° C., head temperature 91° C.,
(7.40 g, 100% purity) swamp temperature 107° C., head temperature 90° C.,
(9.70 g, 100% purity) swamp temperature 109° C., head temperature 92° C.,
(4.10 g, 82% purity) swamp temperature 109° C., head temperature 94° C.,

The fractions with the enriched or pure trans-configured 4-(allyloxymethyl)-2-hexyl-1,3-dioxolane then followed:
(1.90 g, 87% purity) swamp temperature 110° C., head temperature 94° C.,
(0.50 g, 97% purity) swamp temperature 108° C., head temperature 92° C.,
(12.9 g, 100% purity) swamp temperature 108° C., head temperature 92° C.

Analytical and sensory data for the pure cis-configured 4-(allyloxymethyl)-2-hexyl-1,3-dioxolane: ¹³C NMR (101 MHz, CDCl₃) δ 134.9, 116.0, 105.0, 74.7, 71.9, 71.2, 67.5, 34.3, 31.8, 29.3, 24.1, 22.6, 13.9 ppm GC-MS (70 eV): m/z (%)=144 (8), 143 (100), 97 (54), 71 (7), 69 (10), 57 (18), 55 (57), 43 (16), 42 (5), 41 (81), 39 (8), 29 (14). Odor description: Fatty, floral, jasmine, with plastic and rubbery nuances, lacking freshness. The pure cis-isomer has more of an aldehydic cyclamen character in addition to the jasmine note and it recalls faintly plastic. Therefore, it is hedonically less pleasant.

Analytical and sensory data for the pure trans-configured 4-(allyloxymethyl)-2-hexyl-1,3-dioxolane: ¹³C NMR (101 MHz, CDCl₃) δ 134.4, 117.3, 104.7, 74.5, 72.5, 70.5, 67.5, 34.0, 31.7, 29.2, 23.9, 22.5, 14.0. ppm GC-MS (70 eV): m/z (%)=144 (7), 143 (100), 97 (50), 71 (6), 69 (8), 57 (16), 55 (51), 43 (16), 42 (5), 41 (79), 39 (8), 29 (13).

Odor description: Fresh, hesperidic, jasmine, reminiscent of methyl dihydrojasmonate. Compared to the original cis/trans-mixture and the cis-fractions above, the trans-isomer has a more pronounced and complex floral jasmine odor, with a more distinct airy character, with more body and more transparency. Therefore, the trans-isomer is hedonically clearly preferred, especially in its jasmine character and the overall floral transparency. On blotter, the trans-isomer is also distinctly stronger than the cis-isomer, and this becomes even more apparent upon dry down. Accordingly, the trans-isomer lasts longer than the cis-isomer.

### Example 3

### 3.1 Preparation of (2′-hexyl-1′,3′-dioxolan-4′-yl)methanol and 2-hexyl-1,3-dioxan-5-ol, 100% Biobased

pTsOH (5.84 g, 30.2 mmol) was added in portions to a stirred solution of biobased heptanal (178 g, 1.51 mol, supplied by Arkema Deutschland) and biobased glycerol from natural resources (139 g, 1.51 mol, supplied by Mercur Handel GmbH) in cyclohexane (300 ml). The reaction mixture was then heated to reflux in a water separator for 2 h, and subsequently allowed to cool to room temperature. After washing with brine and saturated aq. NaHCO₃ to pH 7, the crude material was dried (Na₂SO₄) and concentrated under reduced pressure. Distillation of the resulting residue (296 g) furnished a colorless cis:trans mixture of (2′-hexyl-1′,3′-dioxolan-4′-yl)methanol and 2-hexyl-1,3-dioxan-5-ol (107 g, 99% purity, 37% yield) (24:21:23:31).

¹³C NMR (101 MHz, C₆D₆): δ 105.3, 76.6, 66.6, 63.5, 34.4, 32.1, 29.6, 24.4, 22.9, 14.3; 105.1, 76.4, 66.8, 62.8, 34.7, 32.1, 29.6, 24.4, 22.9, 14.26; 102.8, 71.8, 71.8, 64.1, 35.4, 32.1, 92.6, 24.2, 23.0, 14.3; 102.2, 71.8, 71.8, 61.5, 34.9, 32.1. 29.6, 24.6, 22.9, 14.3 ppm.

Odor description: Fresh, citric, jasminic, floral, with slightly fatty-metallic facets.

### 3.2 Preparation of 4-(allyloxymethyl)-2-hexyl-1,3-dioxolane and 5-allyloxy-2-hexyl-1,3-dioxane

Under N₂ atmosphere, the solution of the synthesized (2′-hexyl-1′,3′-dioxolan-4′-yl)methanol/2-hexyl-1,3-dioxan-5-ol mixture (10.0 g, 53.1 mmol) was added dropwise with stirring to a suspension of NaH (2.34 g 61.1 mmol) in THF (56 ml). The reaction mixture was heated to reflux for 1 h, and then allowed to cool back to room temperature. At this temperature, 3-chloroprop-1-ene (4.67 g 61.1 mmol) was added dropwise and stirring was continued for further 110 h. The reaction was then quenched with ice-water (25 ml) before being washed to neutrality with aq. NaHCO₃ and brine. After drying (Na₂SO₄) and concentration under reduced pressure the crude product (10.5 g, 81% purity, 70% yield) was obtained, of which an aliquot (3 g) was purified by flash column chromatography (silica gel, cyclohexane/ethyl acetate, 95:5) to provide 4-(allyloxymethyl)-2-hexyl-1,3-dioxolane (0.67 g, 99% purity, R_{I} 1520/1537) as a colorless odoriferous liquid mixture of cis:trans isomers (59:41), and trans 5-allyloxy-2-hexyl-1,3-dioxane (0.42 g, 88% purity, R_{I} 1553) as well as the corresponding cis-isomer (0.49 g, 99% purity, R_{I} 1571).

4-(Allyloxymethyl)-2-hexyl-1,3-dioxolane (cis:trans mixture (59:41)): ¹H NMR (600 MHz, CDCl₃): δ 5.90 (ddt, J=17.0, 10.5, 5.5 Hz, 2H), 5.28 (dq, J=17.0, 1.5 Hz, 1H), 5.28 (dq, J=17.0, 1.5 Hz, 1H), 5.19 (dq, J=10.5, 1.5 Hz, 2H), 4.98 (t, J=5.0 Hz, 1H), 4.89 (t, J=5.0 Hz, 1H), 4.26 (tdd, J=7.0, 6.0, 5.0 Hz, 1H), 4.22 (dtd, J=6.9, 5.9, 5.0 Hz, 1H), 4.13 (dd, J=8.5, 6.5 Hz, 1H), 4.08-3.99 (m, 4H), 3.91 (dd, J=8.5, 7.0 Hz, 1H), 3.77 (dd, J=8.0, 5.0 Hz, 1H), 3.64 (dd, J=8.3, 7.0 Hz, 1H), 3.55 (dd, J=10.0, 5.5 Hz, 1H), 3.53 (dd, J=10.0, 6.0 Hz, 1H), 3.47 (dd, J=10.0, 5.0 Hz, 1H), 3.42 (dd, J=10.0, 6.0 Hz, 1H), 1.74-1.57 (m, 4H), 1.40 (ddt, J=12.0, 8.0, 5.5 Hz, 4H), 1.37-1.21 (m, 12H), 0.88 (t, J=7.0 Hz, 3H), 0.88 (t, J=7.0 Hz, 3H) ppm.

¹³C NMR: δ 134.5, 134.5, 117.4, 117.3, 105.2, 104.7, 74.8, 74.5, 72.5, 72.5, 71.1, 70.5, 67.5, 67.5, 34.0, 34.0, 31.8, 31.8, 29.2, 29.2, 23.9, 23.9, 22.6, 22.6, 14.1, 14.1 ppm. Odor description: Floral, soft, jasminic, reminiscent of methyl dihydrojasmonate, with tea aspects and milky-creamy facets.

cis-Configured 5-allyloxy-2-hexyl-1,3-dioxane isomer: ¹³C NMR: δ 134.9, 117.3, 102.5, 69.8, 69.8, 68.7, 68.7, 34.9, 31.7, 29.1, 24.0, 22.6, 14.1 ppm. Odor description: soft, floral, green, with cardboard and earthy facets.

trans-Configured 5-allyloxy-2-hexyl-1,3-dioxane isomer: ¹³C NMR: δ 134.5, 117.6, 102.2, 70.7, 69.8, 69.8, 68.0, 34.4, 31.7, 29.1, 24.1, 22.5, 14.1 ppm. Odor description: floral, lily-of-the-valley, jasminic, tea, milky, herbal.

### Example 4

### Preparation of (2′-hexyl-1′,3′-dioxolan-4′-yl)methyl acetate and (2′-hexyl-1′,3′-dioxan-5′-yl)acetate

pTsOH (0.14 g, 0.74 mmol) was added in portions to a stirred solution of (2′-hexyl-1′,3′-dioxolan-4′-yl)methanol/2-hexyl-1,3-dioxan-5-ol (7.00 g, 37.2 mmol) from Example 3.1, and acetic acid (2.68 g, 44.6 mmol) in cyclohexane (70 ml). The reaction mixture was heated to reflux in a water separator for 6 h, and subsequently allowed to cool to room temperature. After washing to neutrality, the crude material was dried (Na₂SO₄) and concentrated under reduced pressure to furnish the crude material (7.70 g, 73% purity, 66% yield), of which an aliquot (3 g) was purified by flash column chromatography (silica gel, cyclohexane/ethyl acetate, 92:8) to provide (2′-hexyl-1′,3′-dioxolan-4′-yl)methyl acetate (1.12 g, 99% purity, R_{I} 1528/1541) as a colorless liquid mixture of cis:trans isomers (55:44). (2′-Hexyl-1′,3′-dioxan-5′-yl)acetate: trans-isomer (80 mg, 97% purity, R_{I} 1551) and cis-isomer (130 mg, 99% purity, R_{I} 1539).

trans-(2′-Hexyl-1′,3′-dioxolan-4′-yl)methyl acetate: ¹³C NMR (101 MHz, CDCl₃): 170.9, 104.9, 73.4, 67.0, 64.3, 33.9, 31.7, 29.2, 23.9, 22.6, 20.9, 14.1 ppm. GC-MS (70 eV): m/z (%)=146 (7), 145 (100), 97 (5), 57 (15), 55 (10), 44 (6), 43 (81), 41 (10), 29 (10).

cis-(2′-Hexyl-1′,3′-dioxolan-4′-yl)methyl acetate: ¹³C NMR (101 MHz, CDCl₃): 170.8, 105.5, 73.6, 67.0, 64.9, 33.9, 31.7, 29.2, 23.8, 22.6, 20.9, 14.1 ppm. GC-MS (70 eV): m/z (%)=146 (7), 145 (100), 97 (6), 57 (15), 55 (11), 44 (5), 43 (80), 41 (10), 29 (10).

cis/trans-Mixture of (2′-hexyl-1′,3′-dioxolan-4′-yl)methyl acetate (55/44): Floral, fruity, jasminic, metallic.

trans-(2′-Hexyl-1′,3′-dioxan-5′-yl)acetate: ¹³C NMR (101 MHz, CDCl₃): 169.8, 102.4, 68.3, 68.3, 62.9, 34.3, 31.7, 29.1, 24.0, 22.5, 20.8, 14.1 ppm. GC-MS (70 eV): 145 (54), 117 (7), 113 (6), 97 (8), 86 (5), 57 (12), 55 (7), 43 (100), 41 (9), 29 (9). Odor description: Floral, jasminic, coconut, myrrh, rather weak.

cis-(2′-Hexyl-1′,3′-dioxan-5′-yl)acetate: 171.0, 102.4, 68.7, 68.7, 66.2, 34.9, 31.7, 29.1, 23.9, 22.6, 21.3, 14.1 ppm. GC-MS (70 eV): m/z (%)=146 (5), 145 (69), 117 (7), 97 (7), 86 (5), 57 (14), 55 (7), 44 (5), 43 (100), 41 (9), 29 (9). Odor description: Floral, jasminic, solvent, champignon aspects, metallic undertone.

### Example 5

### Preparation of 4-(ethoxymethyl)-2-hexyl-1,3-dioxolane and 5-ethoxy-2-hexyl-1,3-dioxane

Under N₂ atmosphere, the above synthesized mixture of (2′-hexyl-1′,3′-dioxolan-4′-yl)methanol/2-hexyl-1,3-dioxan-5-ol mixture (10.0 g, 52.4 mmol) from Example 3.1 was added dropwise with stirring to a suspension of NaH (2.31 g 60.2 mmol) in THF (110 ml). The resulting reaction mixture was heated to reflux for 1 h, and then allowed to cool down to room temperature. At this temperature, iodoethane (8.25 g 52.4 mmol) was added dropwise and stirring was continued for a further 91 h. The reaction was then quenched by addition of ice-water (1:1 m 15 ml) before being washed to neutrality with saturated aq. NH₄Cl and brine. After drying (Na₂SO₄) and evaporation of the solvent under reduced pressure, the crude product (9.7 g, 86% purity, 73% yield) was obtained, of which an aliquot (3 g) was purified by flash column chromatography (silica gel, cyclohexane/ethyl acetate, 95:5) to provide a mixture of cis-/trans-configured 4-(ethoxymethyl)-2-hexyl-1,3-dioxolane and 5-ethoxy-2-hexyl-1,3-dioxane (1.48 g, 99% purity, R_{I} 1428/1444/1458) as a colorless odoriferous oil (39:32:29).

cis-Configured 4-(ethoxymethyl)-2-hexyl-1,3-dioxolane isomer: ¹³C NMR: δ 105.2, 74.9, 71.6, 67.5, 67.0, 34.0, 31.76/31.71, 29.23/29.14, 23.9, 22.6, 15.1, 14.08/14.06 ppm. GC-MS (70 eV): m/z (%)=132 (7), 131 (100), 103 (21), 97 (25), 85 (62), 69 (9), 59 (8) 58 (6), 57 (71), 55 (39), 47 (7), 45 (11), 44 (11), 43 (25), 42 (5), 41 (23), 39 (6), 31 (19), 29 (38).

trans-Configured 4-(ethoxymethyl)-2-hexyl-1,3-dioxolane isomer: ¹³C NMR: δ 104.7, 74.6, 71.1, 67.6, 67.1, 34.0, 31.76/31.71, 29.23/29.14, 23.9, 22.6, 15.1, 14.08/14.06 ppm. GC-MS (70 eV): m/z (%)=132 (7), 131 (100), 97 (16), 85 (60), 59 (8), 58 (5), 57 (63), 55 (26), 47 (7), 45 (9), 44 (7), 43 (18), 41 (17), 31 (16), 29 (31).

5-Ethoxy-2-hexyl-1,3-dioxane isomer: ¹³C NMR: δ 102.2, 70.0, 70.0, 68.3, 65.1, 34.5, 31.76/31.71, 29.23/29.14, 24.1, 22.6, 15.6, 14.08/14.06 ppm. GC-MS (70 eV): m/z (%)=131 (65), 85 (28), 73 (17), 72 (100), 59 (5), 57 (35), 55 (13), 45 (12), 44 (54), 43 (42), 41 (16), 31 (9), 29 (31).

Odor description of the mixture: Jasmone, jasmine, lactonic, oak wood, cookie dough.

### Example 6

### Preparation of 2-hexyl-4-(methoxymethyl)-1,3-dioxolane and 2-hexyl-5-methoxy-1,3-dioxane

Under N₂ atmosphere, the solution of the synthesized (2′-hexyl-1′,3′-dioxolan-4′-yl)methanol/2-hexyl-1,3-dioxan-5-ol mixture (10.0 g, 52.4 mmol) was added dropwise with stirring to a suspension of NaH (2.31 g, 60.2 mmol) in THF (110 ml). The resulting reaction mixture was heated to reflux for 1 h and then allowed to cool to room temperature. At this temperature, iodomethane (8.54 g, 60.2 mmol) was added dropwise with stirring, and stirring was continued for further 20 h before the reaction with ice-water (1:1, 15 ml) and washing the crude product mixture to neutrality with saturated aq. NH₄Cl and brine. Drying (Na₂SO₄) and evaporation of the solvent under reduced pressure furnished the crude product (11.0 g, 99% purity, quantitative yield), of which an aliquot (3 g) was purified by flash column chromatography (silica gel, cyclohexane/ethyl acetate, 95:5) to provide 2-hexyl-4-(methoxymethyl)-1,3-dioxolane (650 mg, 100% purity, R_{I} 1362/1378) as a colorless odoriferous liquid mixture of cis:trans isomers (54:46), and trans 2-hexyl-5-methoxy-1,3-dioxane (0.54 g, 99% purity, R_{I} 1388) as well as the corresponding cis-isomer (0.49 g, 98% purity, R_{I}1431).

cis-Configured 2-hexyl-4-(methoxymethyl)-1,3-dioxolane isomer: ¹³C NMR: δ 105.2, 74.7, 73.7, 67.3, 59.4, 33.9, 31.8, 29.2, 23.9, 22.6, 14.1 ppm. GC-MS (70 eV): m/z (%)=118 (5), 117 (100), 97 (12), 71 (83), 45 (20), 43 (16), 41 (24), 29 (17). trans-Configured 2-hexyl-4-(methoxymethyl)-1,3-dioxolane isomer: ¹³C NMR: δ 104.7, 74.5, 73.7, 67.4, 59.4, 34.0, 31.8, 29.2, 23.9, 22.6, 14.1 ppm. GC-MS (70 eV): m/z (%)=118 (5), 117 (100), 97 (9), 71 (83), 57 (8), 55 (19), 45 (19), 43 (15), 41 (23), 31 (6), 29 (15). Odor description of the cis/trans-mixture of 2-hexyl-4-(methoxymethyl)-1,3-dioxolane (53/47): Floral, jasminic, with some metallic and green facets as well as slightly fatty, greasy facets. cis-Configured 2-hexyl-5-methoxy-1,3-dioxane isomer: ¹³C NMR: δ 102.6, 72.5, 68.1, 68.1, 56.5, 34.9, 31.7, 29.1, 24.0, 22.6, 14.1 ppm. GC-MS (70 eV): m/z (%)=117 (69), 71 (38), 59 (26) 58 (100), 57 (7), 55 (6), 45 (8), 43 (21), 41 (16), 31 (5), 29 (16). Odor description: Floral, jasmine, with some aspects of champignons.

trans-Configured 2-hexyl-5-methoxy-1,3-dioxane isomer: ¹³C NMR: δ 102.2, 69.8, 69.6, 69.6, 57.2, 34.4, 31.7, 29.1, 24.1, 22.6, 14.1 ppm. GC-MS (70 eV): m/z (%)=117 (56), 71 (31), 59 (22), 58 (100), 55 (5), 45 (7), 43 (18), 41 (14), 31 (5), 29 (14). Odor description: Floral, metallic, fruity, jasminic, with aspects of cognac.

### Example 7

### Preparation of 2-hexyl-4-(propoxymethyl)-1,3-dioxolane

In a three-neck flask stirring apparatus, 5% palladium on charcoal (1.00 g, 0.21 mmol) was added to a solution of 4-(allyloxymethyl)-2-hexyl-1,3-dioxolane (10.0 g, 42.7 mmol) in tetrahydrofuran (30 ml). The resulting reaction mixture was stirred in a hydrogen atmosphere under ambient pressure for 4 h. The catalyst was removed by filtration, and the reaction mixture concentrated under reduced pressure to afford the crude product (9.71 g, 98% purity, 97% yield). The title compound 2-hexyl-4-(propoxymethyl)-1,3-dioxolane (5.02 g, 97% purity, 51% yield) was obtained by distillation in vacuo at 130° C./0.7 mbar as a colorless odoriferous liquid mixture of cis:trans isomers (57:41).

trans-2-Hexyl-4-(propoxymethyl)-1,3-dioxolane: ¹³C NMR (101 MHz, CDCl₃): 104.9, 74.7, 73.6, 71.4, 67.7, 34.2, 31.9, 29.4, 24.1, 22.9, 22.7, 14.2, 10.6 ppm. GC-MS (70 eV): m/z (%)=146 (7), 145 (86), 99 (29), 97 (19), 69 (6), 58 (7), 57 (100), 55 (26), 47 (5), 45 (7), 44 (8), 43 (58), 42 (5), 41 (28), 39 (5), 31 (5), 29 (17).

cis-2-Hexyl-4-(propoxymethyl)-1,3-dioxolane: ¹³C NMR (101 MHz, CDCl₃): 105.3, 74.9, 73.5, 71.9, 67.6, 34.1, 31.9, 29.4, 24.1, 22.9, 22.7, 14.2, 10.6 ppm. GC-MS (70 eV): m/z (%)=146 (7), 145 (85), 99 (29), 97 (26), 73 (5), 69 (8), 58 (7), 57 (100), 55 (31), 47 (5), 45 (7), 44 (8), 43 (60), 42 (5), 41 (29), 39 (5), 31 (6), 29 (19).

cis/trans-Mixture of 2-hexyl-4-(propoxymethyl)-1,3-dioxolane (57:41): Earthy, champignon, jasminic, metallic, floral-fruity, green.

### Example 8

### Preparation of (2′-pentyl-1′,3′-dioxolan-4′-yl)methanol and 2-pentyl-1,3-dioxan-5-ol

pTsOH (2.22 g, 11.5 mmol) was added in portions to a stirred solution of hexanal (57.5 g, 574 mmol) and renewable glycerol from natural resources (52.9 g, 574 mmol) in cyclohexane (150 ml). The reaction mixture was then heated to reflux in a water separator for 2 h, and subsequently allowed to cool to room temperature. After washing with brine and saturated aq. NaHCO₃ to neutrality, the organic layer was dried (Na₂SO₄) and concentrated under reduced pressure to furnish the crude material (95.4 g, 94% purity, 90% yield), of which an aliquot (3 g) was purified by flash column chromatography (silica gel, cyclohexane/ethyl acetate, 80:20) to provide a cis:trans mixture of (2′-pentyl-1′,3′-dioxolan-4′-yl)methanol and 2-pentyl-1,3-dioxan-5-ol (2.42 g, 99% purity, R_{I} 1275/1294/1257/1323) as a colorless liquid mixture of isomers (19:14:37:29).

¹³C NMR (101 MHz, CDCl₃): δ 105.2, 104.9, 102.8, 102.1, 76.2, 76.1, 71.8, 71.8, 71.5, 71.5, 66.5, 66.4, 64.1, 63.4, 62.7, 61.4, 34.9, 34.3, 34.1, 33.8, 31.7, 31.7, 31.7, 31.7, 23.9, 23.69/23.67, 23.69/23.67, 23.5, 22.6, 22.6, 22.6, 22.6, 14.0, 14.0, 14.0, 14.0 ppm.

Odor description: Floral-jasminic with some metallic and slightly technical aspects.

### Example 9

### 9.1 Preparation of 2-[(2′-hexyl-1′,3′-dioxolan-4′-yl)methoxy]ethanol and 2-[(2′-hexyl-1′,3′-dioxan-5′-yl)oxy]ethanol

Under N₂ atmosphere, the solution of the synthesized methyl 2-[(2′-hexyl-1′,3′-dioxolan-4′-yl)methoxy]acetate/methyl 2-[(2′-hexyl-1′,3′-dioxan-5′-yl)oxy]acetate (7.00 g, 26.9 mmol; Example 10, vide infra) in THF (15 ml) was added dropwise with stirring to a suspension of lithium aluminum hydride (0.83 g 21.5 mmol) in THF (30 ml). The reaction mixture was stirred for 18 h prior to quenching the reaction by addition of isopropanol (5 ml) and 20% sulfuric acid (5 ml). The layers were separated, and the aqueous one was extracted with tert-butyl methyl ether (MTBE, 2×15 ml). The combined organic extracts were washed to neutrality with saturated aq. NH₄Cl and brine. After drying (Na₂SO₄) and evaporation of the solvent under reduced pressure, the resulting crude product (5.82 g, 58% purity, 54% yield) was first distilled in vacuo at 161° C./1.2 mbar. An aliquot (3.3 g) was subsequently purified by flash-column chromatography (silica gel, cyclohexane/ethyl acetate, 81:19) to provide 2-[(2-hexyl-1,3-dioxolan-4-yl)methoxy]ethanol as cis:trans-mixture and cis-configured 2-[(2′-hexyl-1′,3′-dioxan-5′-yl)oxy]ethanol (0.94 g, 98% purity, R_{I} 1648/1666/1686) as a 35:35:28 mixture, and 2-[(2′-hexyl-1′,3′-dioxolan-4′-yl)methoxy]ethanol as cis:trans-mixture as well as trans-configured 2-[(2′-hexyl-1′,3′-dioxan-5′-yl)oxy]ethanol (0.51 g, 100% purity, R_{I} 1648/1666/1691) as a 14:3:83 mixture of isomers.

trans-2-[(2′-hexyl-1′,3′-dioxolan-4′-yl)methoxy]ethanol: ¹³C NMR (101 MHz, CDCl₃): 104.8, 74.7, 72.86/72.81, 71.7, 67.2, 61.8, 34.0, 31.8, 29.2, 24.0, 22.6, 14.1 ppm. GC-MS (70 eV): m/z (%)=148 (6), 147 (95), 101 (76), 97 (21), 73 (7), 71 (6), 69 (9), 59 (7), 58 (10), 57 (100), 55 (35), 47 (6), 45 (68), 44 (8), 43 (26), 42 (5), 41 (25), 39 (6), 31 (14), 29 (30).

cis-2-[(2′-hexyl-1′,3′-dioxolan-4′-yl)methoxy]ethanol: ¹³C NMR (101 MHz, CDCl₃): 105.3, 74.9, 72.86/72.81, 72.2, 67.1, 61.8, 33.9, 31.8, 29.2, 24.0, 22.6, 14.1 ppm. GC-MS (70 eV): m/z (%)=148 (6), 147 (90), 102 (5), 101 (75), 97 (26), 75 (5), 73 (7), 71 (5), 69 (10), 59 (6), 58 (9), 57 (100), 56 (5), 55 (41), 47 (6), 45 (67), 44 (7), 43 (27), 42 (6), 41 (27), 39 (6), 31 (14), 29 (30).

trans-2-[(2′-hexyl-1′,3′-dioxan-5′-yl)oxy]ethanol: ¹³C NMR (101 MHz, CDCl₃): 102.3, 70.6, 69.7, 69.7, 68.9, 62.0, 34.4, 31.7, 29.1, 24.1, 22.6, 14.1 ppm. GC-MS (70 eV): m/z (%)=147 (44), 113 (5), 101 (39), 89 (9), 88 (33), 59 (8), 57 (45), 55 (14), 45 (100), 44 (90), 43 (22), 41 (17), 31 (11), 29 (26).

cis-2-[(2′-hexyl-1′,3′-dioxan-5′-yl)oxy]ethanol: ¹³C NMR (101 MHz, CDCl₃): 102.6, 71.5, 70.1, 68.5, 68.5, 61.9, 34.9, 31.7, 29.1, 24.0, 22.6, 14.1 ppm. GC-MS (70 eV): m/z (%)=147 (47), 113 (5), 101 (41), 89 (10), 88 (31), 87 (5), 75 (6), 69 (6), 59 (9), 58 (5), 57 (51), 55 (18), 45 (100), 44 (82), 43 (25), 41 (19), 31 (12), 29 (27).

Odor description of the isomeric mixture of cis/trans-2-[(2′-hexyl-1′,3′-dioxolan-4′-yl)methoxy]ethanol and trans-configured 2-[(2-hexyl-1,3-dioxan-5-yl)oxy]ethanol (14/3/83): Faintly oily, jasminic, very weak.

Odor description of the cis/trans-mixture of 2-[(2′-hexyl-1′,3′-dioxolan-4′-yl)methoxy]ethanol and cis-configured 2-[(2′-hexyl-1′,3′-dioxan-5′-yl)oxy]ethanol (35/35/28): Slightly floral, citrusy, with green floral facets.

### 9.2 Preparation of 2-[(2′-hexyl-1′,3′-dioxolan-4′-yl)methoxy]acetaldehyde and 2-[(2′-hexyl-1′,3′-dioxan-5′-yl)oxy]acetaldehyde

At 0° C., a solution of potassium bromide (860 mg 7.23 mmol) and tetrabutylammonium bromide (1.17 g, 3.62 mmol) in saturated aq. NaHCO₃ (8.5 ml) was added dropwise to a stirred solution of 2-[(2′-hexyl-1′,3′-dioxolan-4′-yl)methoxy]ethanol and 2-[(2′-hexyl-1′,3′-dioxan-5′-yl)oxy]ethanol (20.6 g, 72.3 mmol) in dichloromethane (400 ml). Next, the 2,2,6,6-tetramethylpiperidinyloxy radical (TEMPO, 560 mg, 3.62 mmol) was added, followed by dropwise addition of a solution of sodium hydrogen carbonate (12.2 g, 145 mmol) in 15% aq. sodium hypochlorite (108 g, 217 mmol). After 1 h of stirring at 0° C., the reaction mixture is allowed to warm to room temperature, and the layers were separated. The organic layer was washed with saturated aq. NH₄Cl solution and brine to neutrality, dried (Na₂SO₄) and concentrated under reduced pressure to provide 2-[(2′-hexyl-1′,3′-dioxolan-4′-yl)methoxy]acetaldehyde/2-[(2′-hexyl-1′,3′-dioxan-5′-yl)oxy]acet-aldehyde (9.83 g, 64% purity, 38% yield), which is used as such without further purification.

### 9.3 Preparation of 1-[(2′-hexyl-1′,3′-dioxolan-4′-yl)methoxy]propan-2-ol and 1-[(2′-hexyl-1′,3′-dioxan-5′-yl)oxy]propan-2-ol

At 0° C. under N₂ atmosphere, a solution of the synthesized 2-[(2′-hexyl-1′,3′-dioxolan-4′-yl)methoxy]acetaldehyde/2-[(2′-hexyl-1′,3′-dioxan-5′-yl)oxy]acet-aldehyde (9.80 g, 42.7 mmol) in THF (10 ml) was added dropwise with stirring to a solution of MeMgCl (3.19 g 42.7 mmol) in THF (65 ml). The resulting reaction mixture was stirred for 30 min. at 0° C., before being allowed to cool down to room temperature. Stirring was continued for further 16 h, before being quenched by pouring into an ice-cold saturated aq. ammonium chloride solution. The aq. layer was extracted twice with tert-butyl methyl ether, and the combined organic layers were washed with brine to neutrality, dried (Na₂SO₄) and evaporated under reduced pressure to furnish the crude product (7.26 g, 56% purity, 39% yield), of which an aliquot (3 g) was purified by flash column chromatography (silica gel, cyclohexane/ethyl acetate, 75:25) to provide cis:trans 1-[(2′-hexyl-1′,3′-dioxolan-4′-yl)methoxy]propan-2-ol, 1-[(2′-hexyl-1′,3′-dioxan-5′-yl)oxy]propan-2-ol (330 mg, 97% purity, R_{I} 1676/1693/1706/1720) as a mixture of cis:trans isomers (47:40:3:7), and cis:trans-configured 1-[(2′-hexyl-1′,3′-dioxolan-4′-yl)methoxy]propan-2-ol, trans-configured 1-[(2′-hexyl-1′,3′-dioxan-5′-yl)oxy]propan-2-ol (430 mg, 87% purity, R_{I} 1676/1693/1720) as a mixture of isomers (16:10:61), and cis-configured 1-[(2′-hexyl-1′,3′-dioxan-5′-yl)oxy]propan-2-ol, trans-configured 1-[(2′-hexyl-1′,3′-dioxolan-4′-yl)methoxy]propan-2-ol (210 mg, 70% purity, R_{I} 1706/1693) as a mixture of isomers (68:2).

cis-Configured 1-[(2′-hexyl-1′,3′-dioxolan-4′-yl)methoxy]propan-2-ol isomer: ¹³C NMR: δ 105.31/105.28, 77.3, 74.83/74.77, 72.27/72.24, 67.15/67.09, 66.4, 33.92/33.90, 31.8, 29.2, 23.99/23.96, 22.6, 18.5, 14.1 ppm. GC-MS (70 eV): m/z (%)=162 (6), 161 (74), 129 (5), 117 (10), 116 (5), 115 (66), 113 (5), 111 (5), 103 (18), 97 (42), 87 (7), 85 (5), 75 (5), 73 (17), 71 (8), 70 (5), 69 (18), 59 (79), 58 (13), 57 (100), 55 (43), 45 (17), 43 (23), 42 (5), 41 (28), 31 (19), 29 (17), 27 (5).

trans-Configured 1-[(2′-hexyl-1′,3′-dioxolan-4′-yl)methoxy]propan-2-ol isomer: ¹³C NMR: δ 104.8, 77.3, 74.65/77.56, 71.83/71.80, 67.26/67.22, 66.4, 34.03/34.00, 31.75/31.71, 29.21/29.13, 24.0, 22.6, 18.5, 14.1 ppm. GC-MS (70 eV): m/z (%)=162 (6), 161 (75), 117 (8), 116 (5), 115 (66), 103 (18), 97 (32), 87 (6), 85 (5), 73 (16), 71 (8), 69 (15), 59 (79), 58 (13), 57 (100), 55 (34), 45 (16), 44 (6), 43 (21), 42 (5), 41 (26), 31 (19), 29 (15).

cis-Configured 1-[(2′-hexyl-1′,3′-dioxan-5′-yl)oxy]propan-2-ol isomer: ¹³C NMR: δ 102.5, 74.5, 71.5, 68.56/68.41, 68.56/68.41, 66.4, 34.9, 31.7, 29.1, 23.9, 22.6, 18.3, 14.1 ppm. GC-MS (70 eV): m/z (%)=171 (17), 161 (36), 117 (6), 115 (33), 113 (8), 111 (33), 103 (10), 102 (11), 97 (13), 87 (7), 85 (6), 75 (7), 73 (16), 71 (6), 69 (30), 59 (62), 58 (100), 57 (73), 55 (28), 45 (20), 44 (7), 43 (26), 41 (24), 31 (17), 29 (17), 27 (5).

trans-Configured 1-[(2′-hexyl-1′,3′-dioxan-5′-yl)oxy]propan-2-ol: ¹³C NMR: δ 102.2, 75.0, 69.71/69.59, 69.71/69.59, 68.9, 66.6, 34.4, 31.7, 29.1, 24.1, 22.5, 18.5, 14.0 ppm. GC-MS (70 eV): m/z (%)=161 (34), 115 (28), 113 (7), 103 (8), 102 (15), 97 (9), 73 (12), 69 (7), 59 (53), 58 (100), 57 (47), 55 (15), 45 (16), 44 (6), 43 (18), 41 (16), 31 (13), 29 (12).

Odor description of a mixture of cis-configured 1-[(2′-hexyl-1′,3′-dioxan-5′-yl)oxy]propan-2-ol and trans-configured 1-[(2′-hexyl-1′,3′-dioxolan-4′-yl)methoxy]propan-2-ol (68/2): Weak floral, jasmonic-oily.

Odor description of cis:trans 1-[(2′-hexyl-1′,3′-dioxolan-4′-yl)methoxy]propan-2-ol and trans-configured 1-[(2′-hexyl-1′,3′-dioxan-5′-yl)oxy]propan-2-ol (16/10/61): Weak, slightly fatty-floral, jasminic.

Odor description of a mixture of cis:trans 1-[(2′-hexyl-1′,3′-dioxolan-4′-yl)methoxy]propan-2-ol and 1-[(2′-hexyl-1′,3′-dioxan-5′-yl)oxy]propan-2-ol (47/40/3/7): Transparent floral, jasmonic, rather weak.

### Example 10

### Preparation of methyl 2-[(2′-hexyl-1′,3′-dioxolan-4′-yl)methoxy]acetate and methyl 2-[(2′-hexyl-1′,3′-dioxan-5′-yl)oxy]acetate

Under N₂ atmosphere, the solution of the synthesized mixture of (2′-hexyl-1′,3′-dioxolan-4′-yl)methanol and 2-hexyl-1,3-dioxan-5-ol (30.0 g, 159 mmol) was added dropwise with stirring to a suspension of NaH (7.02 g 183 mmol) in MTBE (200 ml). The reaction mixture was heated at reflux for 1 h, and then allowed to cool to room temperature. At this temperature, methyl 2-chloroacetate (39.8 g, 183 mmol) was added dropwise with stirring. Stirring was continued for further 30 h, prior to quenching the reaction with ice-water (1:1, 75 ml) and washing the crude product mixture to neutrality with saturated aq. NH₄Cl solution and brine. Drying (Na₂SO₄) and evaporation of the solvent under reduced pressure furnished the crude product (40.9 g, 40% purity, 20% yield), which was first distilled in vacuo at 190° C./1.0 mbar. Subsequently, an aliquot (2.70 g) was purified by flash column chromatography (silica gel, cyclohexane/ethyl acetate, 95:5) to provide a cis:trans mixture of methyl 2-[(2′-hexyl-1′,3′-dioxolan-4′-yl)methoxy]acetate and trans-configured methyl 2-[(2′-hexyl-1′,3′-dioxan-5′-yl)oxy]acetate (370 mg, 80% purity, R_{I} 1720/1741/1757) as a colorless liquid mixture of isomers (29:14:37).

cis-Configured methyl 2-[(2′-hexyl-1′,3′-dioxolan-4′-yl)methoxy]acetate isomer: ¹³C NMR: δ 170.7, 105.3, 74.7, 72.5, 68.6, 67.2, 51.85/52.02, 33.9, 31.7, 29.2, 23.9, 22.54/22.55, 14.1 ppm. GC-MS (70 eV): m/z (%)=179 (6), 176 (7), 175 (98), 130 (7), 129 (100), 97 (22), 91 (6), 87 (8), 74 (5), 73 (16), 71 (83), 69 (10), 59 (9), 58 (7), 57 (41), 55 (36), 45 (33), 44 (6), 43 (32), 41 (28), 39 (6), 31 (13), 29 (33).

trans-Configured methyl 2-[(2′-hexyl-1′,3′-dioxolan-4′-yl)methoxy]acetate isomer: ¹³C NMR: δ 170.7, 104.8, 74.4, 71.9, 68.6, 67.3, 51.85/52.02, 34.0, 31.7, 29.2, 23.9, 22.54/22.55, 14.1 ppm. GC-MS (70 eV): m/z (%)=176 (7), 175 (95), 130 (7), 129 (100), 97 (19), 91 (5), 87 (8), 74 (5), 73 (15), 72 (5), 71 (85), 69 (9), 59 (8), 58 (8), 57 (43), 55 (35), 45 (35), 44 (7), 43 (34), 42 (9), 41 (30), 39 (7), 31 (15), 29 (33).

trans-Configured methyl 2-[(2′-hexyl-1′,3′-dioxan-5′-yl)oxy]acetate isomer: ¹³C NMR: δ 170.5, 102.2, 69.9, 69.3, 69.3, 67.1, 51.85/51.02, 34.4, 31.7, 29.1, 24.1, 22.54/22.55, 14.1 ppm. GC-MS (70 eV): m/z (%)=259 (5), 176 (7), 175 (91), 147 (5), 129 (12), 119 (12), 117 (38), 116 (100), 113 (8), 101 (5), 88 (25), 87 (8), 85 (5), 75 (14), 74 (9), 73 (13), 71 (43), 69 (9), 59 (27), 58 (27), 57 (72), 55 (21), 45 (31), 44 (7), 43 (52), 42 (44), 41 (32), 39 (7), 31 (23), 29 (55).

Odor description of cis/trans methyl 2-[(2′-hexyl-1′,3′-dioxolan-4′-yl)methoxy]acetate and trans-configured methyl 2-[(2′-hexyl-1′,3′-dioxan-5′-yl)oxy]acetate (29/14/37): Jasmine lactone, creamy, coconut, lactonic, with nutty nuances in the direction of hazelnut.

### Example 11

### Preparation of a mixture of 1-[(2′-hexyl-1′,3′-dioxolan-4′-yl)methoxy]propan-2-one and 1-[(2′-hexyl-1′,3′-dioxan-5′-yl)oxy]propan-2-one

At 0° C., a solution of potassium bromide (0.20 g, 1.70 mmol) and tetrabutylammonium bromide (0.27 g 0.85 mmol) in saturated aq. NaHCO₃ (1.1 ml) was added dropwise with stirring to a solution of 1-[(2′-hexyl-1′,3′-dioxolan-4′-yl)methoxy]propan-2-ol and 1-[(2′-hexyl-1′,3′-dioxan-5′-yl)oxy]propan-2-ol (4.20 g, 17.1 mmol) in dichloromethane (50 ml). Then, 2,2,6,6-tetramethylpiperidinyloxy radical (TEMPO, 130 mg, 0.85 mmol) was added, followed by dropwise addition of a solution of sodium hydrogen carbonate (2.86 g, 34.1 mmol) in 15% aq. sodium hypochlorite (25.4 g, 51.2 mmol). After 1 h of stirring at 0° C., the reaction mixture was allowed to warm to room temperature, and the layers were separated.

The organic layer was washed with saturated aq. NH₄Cl solution and brine to neutrality, dried (Na₂SO₄) and concentrated under reduced pressure to provide the crude product (3.71 g, 52% purity, 46% yield), which was purified by flash-column chromatography (silica gel, cyclohexane/ethyl acetate, 88:12) to provide the cis:trans-configured 1-[(2′-hexyl-1′,3′-dioxolan-4′-yl)methoxy]propan-2-one and the trans-configured 1-[(2′-hexyl-1′,3′-dioxan-5′-yl)oxy]propan-2-one (270 mg, 94% purity, R_{I} 1681/1699/1720) as an isomeric mixture (35:20:39) as well as pure trans-configured 1-[(2′-hexyl-1′,3′-dioxan-5′-yl)oxy]propan-2-one (200 mg, 98% purity, R_{I} 1720).

### cis-configured 1-[(2′-hexyl-1′,3′-dioxolan-4′-yl)methoxy]propan-2-one

¹³C NMR: δ 206.6, 105.3, 76.8, 74.8, 72.4, 67.1, 34.0, 31.8, 29.2, 26.3, 24.0, 22.6, 14.1 ppm. GC-MS (70 eV): m/z (%)=160 (8), 159 (100), 114 (7), 113 (100), 99 (8), 97 (27), 87 (11), 85 (9), 75 (10), 73 (5), 71 (8), 69 (13), 58 (10), 57 (88), 55 (33), 45 (10), 43 (53), 42 (5), 41 (23), 31 (8), 29 (24), 27 (7).

### trans-configured 1-[(2′-hexyl-1′,3′-dioxolan-4′-yl)methoxy]propan-2-one

13C NMR: δ 206.5, 104.9, 76.8, 74.5, 71.9, 67.2, 34.0, 31.8, 29.2, 26.3, 23.9, 22.6, 14.1 ppm. GC-MS (70 eV): m/z (%)=160 (7), 159 (100), 114 (7), 113 (98), 99 (8), 97 (18), 87 (8), 85 (9), 75 (8), 71 (7), 69 (11), 58 (9), 57 (81), 55 (26), 45 (8), 43 (48), 41 (21), 31 (7), 29 (22), 27 (6).

### cis-configured 1-[(2′-hexyl-1′,3′-dioxan-5′-yl)oxy]propan-2-one

¹³C NMR: δ 207.4, 102.5, 74.1, 71.3, 68.5, 68.5, 34.9, 31.7, 29.1, 26.5, 23.9, 22.6, 14.1 ppm. GC-MS (70 eV): m/z (%)=160 (6), 159 (70), 113 (53), 111 (5), 101 (8), 100 (22), 99 (7), 97 (7), 87 (23), 85 (8), 75 (10), 73 (6), 72 (26), 71 (9), 70 (7), 69 (23), 59 (7), 58 (16), 57 (100), 55 (33), 45 (15), 44 (6), 43 (90), 42 (22), 41 (23), 31 (13), 29 (41), 27 (12).

### trans-configured 1-[(2′-hexyl-1′,3′-dioxan-5′-yl)oxy]propan-2-one

¹³C NMR: δ 205.6, 102.3, 74.9, 69.7, 69.4, 69.4, 34.4, 31.7, 29.1, 26.2, 24.1, 22.6, 14.1 ppm. GC-MS (70 eV): m/z (%)=160 (8), 159 (98), 114 (6), 113 (77), 101 (18), 100 (54), 99 (7), 97 (5), 87 (5), 85 (10), 75 (10), 73 (6), 72 (55), 71 (8), 70 (8), 69 (13), 59 (8), 58 (20), 57 (72), 55 (21), 45 (7), 44 (5), 43 (100), 42 (40), 41 (19), 31 (11), 29 (39), 27 (10).

cis/trans-1-[(2′-hexyl-1′,3′-dioxolan-4′-yl)methoxy]propan-2-one and trans-configured 1-[(2′-hexyl-1′,3′-dioxan-5′-yl)oxy]propan-2-one (35:20:39). Odor description: fresh aldehydic, transparent white floral, jasminic, slightly metallic.

trans-configured 1-[(2′-hexyl-1′,3′-dioxan-5′-yl)oxy]propan-2-one. Odor description: fresh aldehydic, transparent white floral, jasminic, lily of the valley (muguet) with Florhydral aspects and some facets of green banana.

### Example 12: All-Purpose Cleaner, pH Neutral

TABLE 1

| | Perfume | | | Perfume | |
|---|---|---|---|---|---|
| | oil 1 | Perfume | Perfume | oil 4 | Perfume |
| | (not | oil 2 | oil 3 | (not | oil 5 |
| | according | (according | (according | according | (according |
| | to | to | to | to | to |
| Ingredients | invention) | invention) | invention) | invention) | invention) |
| EUCALYPTUS OIL | 11.000 | 11.000 | 11.000 | 11.000 | 11.000 |
| GLOBULUS (CAS | | | | | |
| NO. 8000-48-4) | | | | | |
| CLOVE BUD OIL | 1.500 | 1.500 | 1.500 | 1.500 | 1.500 |
| (CAS NO. 8000-34-8) | | | | | |
| ORANGE OIL | 18.000 | 18.000 | 18.000 | 18.000 | 18.000 |
| TERPENES (CAS | | | | | |
| NO. 68647-72-3) | | | | | |
| CEDARWOOD | 2.500 | 2.500 | 2.500 | 2.500 | 2.500 |
| CHINESE OIL (CAS | | | | | |
| NO. 85085-29-6) | | | | | |
| DIHYDRO | 130.000 | 130.000 | 130.000 | 130.000 | 130.000 |
| MYRCENOL (2,6- | | | | | |
| DIMETHYLOCT-7- | | | | | |
| EN-2-OL, CAS NO. | | | | | |
| 18479-58-8) | | | | | |
| HERBAFLORAT | 45.000 | 45.000 | 45.000 | 45.000 | 45.000 |
| (TRICYCLO[5.2.1.0²,⁶] | | | | | |
| DEC-3-EN-8-YL | | | | | |
| ACETATE CAS, NO. | | | | | |
| 5413-60-5) | | | | | |
| HEXYL SALICYLATE | 14.000 | 14.000 | 14.000 | 14.000 | 14.000 |
| (CAS NO. 6259-76-3) | | | | | |
| GERANIOL (3,7- | 18.000 | 18.000 | 18.000 | 18.000 | 18.000 |
| DIMETHYLOCTA-2,6- | | | | | |
| DIEN-1-OL, CAS NO. | | | | | |
| 106-24-1) | | | | | |
| DIMETHYL BENZYL | 3.000 | 3.000 | 3.000 | 3.000 | 3.000 |
| CARBINYL | | | | | |
| BUTYRATE (1,1- | | | | | |
| DIMETHYL-2- | | | | | |
| PHENYLETHYL | | | | | |
| BUTYRATE, CAS | | | | | |
| NO. 10094-34-5) | | | | | |
| AMYL SALICYLATE | 45.000 | 45.000 | 45.000 | 45.000 | 45.000 |
| (PENTYL 2- | | | | | |
| HYDROXYBENZOATE, | | | | | |
| CAS NO. 2050-08-0) | | | | | |
| PHENIRAT ® (2- | 9.000 | 9.000 | 9.000 | 9.000 | 9.000 |
| PHENOXYETHYL 2- | | | | | |
| METHYLPROPANOATE, | | | | | |
| CAS NO. 103-60-6) | | | | | |
| GLOBALIDE ® | 26.000 | 26.000 | 26.000 | 26.000 | 26.000 |
| (((12E)-1- | | | | | |
| OXACYCLOHEXADE | | | | | |
| C-12-EN-2-ONE; | | | | | |
| (12Z)-1- | | | | | |
| OXACYCLOHEXADE | | | | | |
| C-12-EN-2-ONE; | | | | | |
| (13E)-1- | | | | | |
| OXACYCLOHEXADE | | | | | |
| C-13-EN-2-ONE; (13Z)-1- | | | | | |
| OXACYCLOHEXADE | | | | | |
| C-13-EN-2-ONE)), | | | | | |
| CAS NO. 111879-80-2) | | | | | |
| ALDEHYDE C12 | 3.000 | 3.000 | 3.000 | 3.000 | 3.000 |
| LAURIC | | | | | |
| (DODECANAL, CAS | | | | | |
| NO. 112-54-9) | | | | | |
| COUMARIN (2H- | 4.000 | 4.000 | 4.000 | 4.000 | 4.000 |
| CHROMEN-2-ONE, | | | | | |
| CAS NO. 91-64-5) | | | | | |
| DIPHENYL OXIDE | 5.000 | 5.000 | 5.000 | 5.000 | 5.000 |
| (PHENOXYBENZENE, | | | | | |
| CAS NO. 101-84-8) | | | | | |
| ISOBORNYL | 120.000 | 120.000 | 120.000 | 120.000 | 120.000 |
| ACETATE (1,7,7- | | | | | |
| TRIMETHYLBICYCLO | | | | | |
| [2.2.1]HEPT-2-YL | | | | | |
| ACETATE, CAS NO. | | | | | |
| 125-12-2) | | | | | |
| CAMPHOR DL (1,7,7- | 3.500 | 3.500 | 3.500 | 3.500 | 3.500 |
| TRIMETHYLBICYCLO | | | | | |
| [2.2.1]HEPTAN-2- | | | | | |
| ONE, CAS NO. 76- | | | | | |
| 22-2) | | | | | |
| LINALOOL (3,7- | 40.000 | 40.000 | 40.000 | 40.000 | 40.000 |
| DIMETHYLOCTA-1,6- | | | | | |
| DIEN-3-OL, CAS NO. | | | | | |
| 78-70-6) | | | | | |
| PHENYLETHYL | 14.000 | 14.000 | 14.000 | 14.000 | 14.000 |
| ALCOHOL (2- | | | | | |
| PHENYLETHANOL, | | | | | |
| CAS NO. 60-12-8) | | | | | |
| TERPINEOL PURE | 39.000 | 39.000 | 39.000 | 39.000 | 39.000 |
| (REACTION MASS | | | | | |
| OF α,α-4- | | | | | |
| TRIMETHYL-(1S)-3- | | | | | |
| CYCLOHEXENE-1- | | | | | |
| METHANOL AND | | | | | |
| α,α-4-TRIMETHYL- | | | | | |
| (1R)-3- | | | | | |
| CYCLOHEXENE-1- | | | | | |
| METHANOL AND 1- | | | | | |
| METHYL-4-(1- | | | | | |
| METHYLETHYLIDENE)- | | | | | |
| CYCLOHEXANOL, | | | | | |
| CAS NO. 8000-41-7) | | | | | |
| TERPINYL ACETATE | 22.000 | 22.000 | 22.000 | 22.000 | 22.000 |
| (REACTION MASS | | | | | |
| OF 1-METHYL-4- | | | | | |
| (PROPAN-2- | | | | | |
| YLIDENE)CYCLOHEXYL | | | | | |
| ACETATE AND | | | | | |
| 2-(4- | | | | | |
| METHYLCYCLOHEX- | | | | | |
| 3-EN-1-YL)PROPAN- | | | | | |
| 2-YL ACETATE, CAS | | | | | |
| NO. 8007-35-0) | | | | | |
| AGRUNITRIL (3,7- | 6.000 | 6.000 | 6.000 | 6.000 | 6.000 |
| DIMETHYLOCT-6- | | | | | |
| ENENITRILE, CAS | | | | | |
| NO. 51566-62-2) | | | | | |
| METHYL IONONE 70 | 9.000 | 9.000 | 9.000 | 9.000 | 9.000 |
| (((1E)-1-(2,6,6- | | | | | |
| TRIMETHYLCYCLOH | | | | | |
| EX-2-EN-1-YL)PENT- | | | | | |
| 1-EN-3-ONE; (3E)-3- | | | | | |
| METHYL-4-(2,6,6- | | | | | |
| TRIMETHYL- | | | | | |
| CYCLOHEX-2-EN-1- | | | | | |
| YL)BUT-3-EN-2- | | | | | |
| ONE), CAS NO. | | | | | |
| 1335-46-2) | | | | | |
| ALLYL AMYL | 3.500 | 3.500 | 3.500 | 3.500 | 3.500 |
| GLYCOLATE (ALLYL | | | | | |
| (3-METHYLBUTOXY) | | | | | |
| ACETATE, CAS NO. | | | | | |
| 67634-00-8) | | | | | |
| CITRAL (3,7- | 4.500 | 4.500 | 4.500 | 4.500 | 4.500 |
| DIMETHYLOCTA-2,6- | | | | | |
| DIENAL, CAS NO. | | | | | |
| 5392-40-5) | | | | | |
| ALDEHYDE C12 | 9.000 | 9.000 | 9.000 | 9.000 | 9.000 |
| MNA (2- | | | | | |
| METHYLUNDECANAL, | | | | | |
| CAS NO. 110-41-8) | | | | | |
| ALCOHOL C10 | 22.000 | 22.000 | 22.000 | 22.000 | 22.000 |
| (DECAN-1-OL, CAS | | | | | |
| NO. 112-30-1) | | | | | |
| MARITIMA 1% DPG | 2.000 | 2.000 | 2.000 | 2.000 | 2.000 |
| (4-[(3E)-4,8- | | | | | |
| DIMETHYLNONA- | | | | | |
| 3,7- | | | | | |
| DIENYL]PYRIDINE, | | | | | |
| CAS NO. 38462-23-6) | | | | | |
| FARENAL ® 10% | 4.500 | 4.500 | 4.500 | 4.500 | 4.500 |
| DPG (2,6,10- | | | | | |
| TRIMETHYLUNDEC- | | | | | |
| 9-ENAL, CAS NO. | | | | | |
| 141-13-9) | | | | | |
| EVERNYL 10% DPG | 4.500 | 4.500 | 4.500 | 4.500 | 4.500 |
| (METHYL 2,4- | | | | | |
| DIHYDROXY-3,6- | | | | | |
| DIMETHYLBENZOATE, | | | | | |
| CAS NO. 4707-47-5) | | | | | |
| VERTOCITRAL (2,4- | 3.000 | 3.000 | 3.000 | 3.000 | 3.000 |
| DIMETHYLCYCLOHE | | | | | |
| X-3-ENE-1- | | | | | |
| CARBALDEHYDE, | | | | | |
| CAS NO. 68039-49-6) | | | | | |
| AGRUMEX (2-TERT- | 15.000 | 15.000 | 15.000 | 15.000 | 15.000 |
| BUTYLCYCLOHEXYL | | | | | |
| ACETATE, CAS | | | | | |
| NO. 20298-69-5) | | | | | |
| ALDEHYDE C14 SO- | 3.000 | 3.000 | 3.000 | 3.000 | 3.000 |
| CALLED (5- | | | | | |
| HEPTYLOXOLAN-2- | | | | | |
| ONE, CAS NO. 104- | | | | | |
| 67-6) | | | | | |
| ALLYL CAPROATE | 3.500 | 3.500 | 3.500 | 3.500 | 3.500 |
| (2-PROPENYL | | | | | |
| HEXANOATE, CAS | | | | | |
| NO. 123-68-2) | | | | | |
| HEXYL ACETATE | 2.000 | 2.000 | 2.000 | 2.000 | 2.000 |
| (CAS NO. 142-92-7) | | | | | |
| STYRALYL | 8.000 | 8.000 | 8.000 | 8.000 | 8.000 |
| ACETATE (1- | | | | | |
| PHENYLETHYL | | | | | |
| ACETATE, CAS NO. | | | | | |
| 93-92-5) | | | | | |
| CITRONITRILE ((2E)- | 7.000 | 7.000 | 7.000 | 7.000 | 7.000 |
| 3-METHYL-5- | | | | | |
| PHENYLPENT-2- | | | | | |
| ENENITRILE, CAS | | | | | |
| NO. 53243-59-7) | | | | | |
| BENZYL ACETATE | 22.000 | 22.000 | 22.000 | 22.000 | 22.000 |
| (CAS NO. 140-11-4) | | | | | |
| ISOMENTHONE (2- | 10.000 | 10.000 | 10.000 | 10.000 | 10.000 |
| ISOPROPYL-5- | | | | | |
| METHYLCYCLOHEXANONE, | | | | | |
| CAS NO. 10458-14-7) | | | | | |
| ORYCLON SPECIAL | 20.000 | 20.000 | 20.000 | 20.000 | 20.000 |
| (4-TERT- | | | | | |
| BUTYLCYCLOHEXYL | | | | | |
| ACETATE, CAS | | | | | |
| NO. 32210-23-4) | | | | | |
| NEROLIN YARA | 5.000 | 5.000 | 5.000 | 5.000 | 5.000 |
| YARA CRIST. (2- | | | | | |
| METHOXYNAPHTHALENE, | | | | | |
| CAS NO. 93-04-9) | | | | | |
| FLOROPAL (2,4,6- | 3.500 | 3.500 | 3.500 | 3.500 | 3.500 |
| TRIMETHYL-4- | | | | | |
| PHENYL-1,3- | | | | | |
| DIOXANE, CAS NO. | | | | | |
| 5182-36-5) | | | | | |
| ALLYL | 2.000 | 2.000 | 2.000 | 2.000 | 2.000 |
| CYCLOHEXYL | | | | | |
| PROPIONATE | | | | | |
| (PROP-2-EN-1-YL 3- | | | | | |
| CYCLOHEXYLPROPANOATE, | | | | | |
| CAS NO. 2705-87-5) | | | | | |
| PATCHOULI OIL | 3.500 | 3.500 | 3.500 | 3.500 | 3.500 |
| (CAS NO. 8014-09-3) | | | | | |
| NEROLEX (3,7- | 9.000 | 9.000 | 9.000 | 9.000 | 9.000 |
| DIMETHYLOCTA-2,6- | | | | | |
| DIEN-1-OL, CAS NO. | | | | | |
| 106-25-2) | | | | | |
| CYCLODUMOL | 70.000 | 70.000 | 70.000 | 70.000 | 70.000 |
| ACETATE (1-(3,3- | | | | | |
| DIMETHYLCYCLOHEXYL) | | | | | |
| ETHYL ACETATE, | | | | | |
| CAS NO. 25225-10-9) | | | | | |
| DIPROPYLENE | 175.000 | 145.000 | 145.000 | 145.000 | 145.000 |
| GLYCOL (1,1-OXYDI- | | | | | |
| 2-PROPANOL, CAS | | | | | |
| NO. 25265-71-8) | | | | | |
| METHYL | 0.000 | 0.000 | 0.000 | 30.000 | 0.000 |
| DIHYDROJASMONATE | | | | | |
| (CAS NO. 24851-98-7) | | | | | |
| Product of Example 1 | 0.000 | 30.000 | 0.000 | 0.000 | 0.000 |
| (cis: trans mixture, | | | | | |
| 55:45) | | | | | |
| Product of Example | 0.000 | 0.000 | 30.000 | 0.000 | 0.000 |
| 2.1 (cis: trans mixture, | | | | | |
| 61:38) | | | | | |
| Product of Example 8 | 0.000 | 0.000 | 0.000 | 0.000 | 30.000 |
| | 1000.000 | 1000.000 | 1000.000 | 1000.000 | 1000.000 |

Perfume oils 1 to 5 were evaluated in all-purpose cleaner, pH neutral, at a concentration of 0.3%, respectively, by a panel of trained perfumers. The samples of the all-purpose cleaners were olfactorily evaluated in blind by the panel against a benchmark (methyl dihydrojasmonate in perfume oil 4) and an odorless solvent (DPG) (perfume oil 1).

The products of Examples 1, 2.1, and 8 all enhanced the floral-jasmine note compared to the version without. The effect is similar to the floral-jasmine effect of methyl dihydrojasmonate yet providing more transparent floralcy. The product of Example 1 and the product of Example 2.1 improve more the floral freshness in a similar way to methyl dihydrojasmonate.

### Example 13: Body Lotion

TABLE 2

| | Perfume | Perfume | | | |
|---|---|---|---|---|---|
| | oil 6 | oil 7 | Perfume | Perfume | Perfume |
| | (not | (not | oil 8 | oil 9 | oil 10 |
| | according | according | (according | (according | (according |
| | to | to | to | to | to |
| Ingredients | invention) | invention) | invention) | invention) | invention) |
| EUCALYPTUS OIL | 1.000 | 1.000 | 1.000 | 1.000 | 1.000 |
| GLOBULUS (CAS | | | | | |
| NO. 8000-48-4) | | | | | |
| HEXYL SALICYLATE | 8.000 | 8.000 | 8.000 | 8.000 | 8.000 |
| (CAS NO. 6259-76-3) | | | | | |
| CYCLOHEXYL | 5.000 | 5.000 | 5.000 | 5.000 | 5.000 |
| SALICYLATE | | | | | |
| (CYCLOHEXYL 2- | | | | | |
| HYDROXYBENZOATE, | | | | | |
| CAS NO. 25485- | | | | | |
| 88-5) | | | | | |
| ALDEHYDE C10 | 1.000 | 1.000 | 1.000 | 1.000 | 1.000 |
| (DECANAL CAS, NO. | | | | | |
| 112-31-2) | | | | | |
| DELPHONE (2- | 2.000 | 2.000 | 2.000 | 2.000 | 2.000 |
| PENTYLCYCLOPEN | | | | | |
| TAN-1-ONE, CAS | | | | | |
| NO. 4819-67-4) | | | | | |
| ALDEHYDE C18 SO- | 2.000 | 2.000 | 2.000 | 2.000 | 2.000 |
| CALLED (5- | | | | | |
| PENTYLOXOLAN-2- | | | | | |
| ONE, CAS NO. 104- | | | | | |
| 61-0) | | | | | |
| PHENYLETHYL | 8.000 | 8.000 | 8.000 | 8.000 | 8.000 |
| PHENYLACETATE | | | | | |
| (2-PHENYLETHYL 2- | | | | | |
| PHENYLACETATE, | | | | | |
| CAS NO. 102-20-5) | | | | | |
| MENTHANYL | 5.000 | 5.000 | 5.000 | 5.000 | 5.000 |
| ACETATE | | | | | |
| (REACTION MASS | | | | | |
| OF CIS-1-METHYL-1-(4- | | | | | |
| METHYLCYCLOHEXYL) | | | | | |
| ETHYL | | | | | |
| ACETATE AND | | | | | |
| TRANS-1-METHYL-1-(4- | | | | | |
| METHYLCYCLOHEXYL) | | | | | |
| ETHYL | | | | | |
| ACETATE AND CIS- | | | | | |
| 4-ISOPROPYL-1- | | | | | |
| METHYLCYCLOHEXYL | | | | | |
| ACETATE AND | | | | | |
| TRANS-4- | | | | | |
| ISOPROPYL-1- | | | | | |
| METHYLCYCLOHEXYL | | | | | |
| ACETATE, NO | | | | | |
| CAS NUMBER) | | | | | |
| AMYL SALICYLATE | 4.000 | 4.000 | 4.000 | 4.000 | 4.000 |
| (PENTYL 2- | | | | | |
| HYDROXYBENZOATE, | | | | | |
| CAS NO. 2050-08-0) | | | | | |
| MAJANTOL ® (3-(2,2- | 13.000 | 13.000 | 13.000 | 13.000 | 13.000 |
| DIMETHYL-3- | | | | | |
| HYDROXYPROPYL) | | | | | |
| TOLUENE, CAS NO. | | | | | |
| 103694-68-4) | | | | | |
| ETHYL LINALYL | 5.000 | 5.000 | 5.000 | 5.000 | 5.000 |
| ACETATE ((6E)-3,7- | | | | | |
| DIMETHYLNONA- | | | | | |
| 1,6-DIEN-3-YL | | | | | |
| ACETATE, CAS NO. | | | | | |
| 61931-80-4) | | | | | |
| PHENIRAT ® (2- | 20.000 | 20.000 | 20.000 | 20.000 | 20.000 |
| PHENOXYETHYL 2- | | | | | |
| METHYLPROPANOATE, | | | | | |
| CAS NO. 103-60-6) | | | | | |
| GLOBALIDE ® | 32.000 | 32.000 | 32.000 | 32.000 | 32.000 |
| (((12E)-1- | | | | | |
| OXACYCLOHEXADE | | | | | |
| C-12-EN-2-ONE; | | | | | |
| (12Z)-1- | | | | | |
| OXACYCLOHEXADE | | | | | |
| C-12-EN-2-ONE; | | | | | |
| (13E)-1- | | | | | |
| OXACYCLOHEXADE | | | | | |
| C-13-EN-2-ONE; | | | | | |
| (13Z)-1- | | | | | |
| OXACYCLOHEXADE | | | | | |
| C-13-EN-2-ONE)), | | | | | |
| CAS NO. 111879-80-2) | | | | | |
| TETRAHYDROGERANIOL | 5.000 | 5.000 | 5.000 | 5.000 | 5.000 |
| (3,7- | | | | | |
| DIMETHYLOCTAN-2- | | | | | |
| OL, CAS NO. 106-21-8) | | | | | |
| PEONILE (2- | 3.000 | 3.000 | 3.000 | 3.000 | 3.000 |
| CYCLOHEXYLIDENE-2- | | | | | |
| PHENYLACETONITRILE, | | | | | |
| CAS NO. 10461- | | | | | |
| 98-0) | | | | | |
| ALDEHYDE C12 | 0.500 | 0.500 | 0.500 | 0.500 | 0.500 |
| LAURIC | | | | | |
| (DODECANAL, CAS | | | | | |
| NO. 112-54-9) | | | | | |
| ANISIC ALDEHYDE | 8.000 | 8.000 | 8.000 | 8.000 | 8.000 |
| PURE (4- | | | | | |
| METHOXYBENZALDEHYDE, | | | | | |
| CAS NO. | | | | | |
| 123-11-5) | | | | | |
| ETHYL VANILLIN (3- | 3.000 | 3.000 | 3.000 | 3.000 | 3.000 |
| ETHOXY-4- | | | | | |
| HYDROXYBENZALD | | | | | |
| EHYDE, CAS NO. | | | | | |
| 121-32-4) | | | | | |
| DIMETHYL BENZYL | 5.000 | 5.000 | 5.000 | 5.000 | 5.000 |
| CARBINYL ACETATE | | | | | |
| (1,1-DIMETHYL-2- | | | | | |
| PHENYLETHYL | | | | | |
| ACETATE, CAS NO. | | | | | |
| 151-05-3) | | | | | |
| PHENYLETHYL | 25.000 | 25.000 | 25.000 | 25.000 | 25.000 |
| ALCOHOL (2- | | | | | |
| PHENYLETHANOL, | | | | | |
| CAS NO. 60-12-8) | | | | | |
| VANILLIN (4- | 1.000 | 1.000 | 1.000 | 1.000 | 1.000 |
| HYDROXY-3- | | | | | |
| METHOXYBENZALDEHYDE, | | | | | |
| CAS NO. | | | | | |
| 121-33-5) | | | | | |
| ETHYLENE | 180.000 | 180.000 | 180.000 | 180.000 | 180.000 |
| BRASSYLATE (1,4- | | | | | |
| DIOXACYCLOHEPTA | | | | | |
| DECANE-5,17- | | | | | |
| DIONE, CAS NO. | | | | | |
| 105-95-3) | | | | | |
| LINALYL | 3.000 | 3.000 | 3.000 | 3.000 | 3.000 |
| PROPIONATE (3,7- | | | | | |
| DIMETHYLOCTA-1,6- | | | | | |
| DIEN-3-YL | | | | | |
| PROPANOATE, CAS | | | | | |
| NO. 144-39-8) | | | | | |
| CEDRYL ACETATE | 5.000 | 5.000 | 5.000 | 5.000 | 5.000 |
| (REACTION MASS | | | | | |
| FROM THE | | | | | |
| ESTERIFICATION | | | | | |
| OF CEDROL RICH | | | | | |
| CEDARWOOD OIL | | | | | |
| FRACTIONS), CAS | | | | | |
| NO. 77-54-3) | | | | | |
| ETHYL LINALOOL | 12.000 | 12.000 | 12.000 | 12.000 | 12.000 |
| (3,7-DIMETHYLNONA- | | | | | |
| 1,6-DIEN-3-OL CAS | | | | | |
| NO. 10339-55-6) | | | | | |
| ISO E SUPER | 20.000 | 20.000 | 20.000 | 20.000 | 20.000 |
| (REACTION MASS | | | | | |
| FROM THE ACIDIC | | | | | |
| CYCLIZATION OF 1- | | | | | |
| (1′,6′-DIMETHYL-4′- | | | | | |
| (4″-METHYLPENT- | | | | | |
| 3″-EN-1″- | | | | | |
| YL)CYCLOHEX-3′- | | | | | |
| EN-1′-YL)ETHAN-1- | | | | | |
| ONE, CAS NO. | | | | | |
| 54464-57-2) | | | | | |
| IONONE BETA (4- | 13.000 | 13.000 | 13.000 | 13.000 | 13.000 |
| (2,6,6- | | | | | |
| TRIMETHYLCYCLOH | | | | | |
| EX-1-EN-1-YL)BUT- | | | | | |
| 3-EN-2-ONE, CAS | | | | | |
| NO. 79-77-6) | | | | | |
| IONONE ALPHA (4- | 11.000 | 11.000 | 11.000 | 11.000 | 11.000 |
| (2,6,6- | | | | | |
| TRIMETHYLCYCLOH | | | | | |
| EX-2-ENE-1-YL)- | | | | | |
| BUT-3-ENE-2-ONE, | | | | | |
| CAS NO. 127-41-3) | | | | | |
| PHENOXANOL (3- | 25.000 | 25.000 | 25.000 | 25.000 | 25.000 |
| METHYL-5- | | | | | |
| PHENYLPENTAN-1- | | | | | |
| OL, CAS NO. 55066- | | | | | |
| 48-3) | | | | | |
| ETHYL SAFRANATE | 2.000 | 2.000 | 2.000 | 2.000 | 2.000 |
| 10% DPG (ETHYL | | | | | |
| 2,6,6- | | | | | |
| TRIMETHYLCYCLOH | | | | | |
| EXA-1,3-DIENE-1- | | | | | |
| CARBOXYLATE, | | | | | |
| CAS NO. 35044-59-8) | | | | | |
| HEXENYL ACETATE | 2.500 | 2.500 | 2.500 | 2.500 | 2.500 |
| CIS-3 10% DPG ((Z)- | | | | | |
| HEX-3-ENYL | | | | | |
| ACETATE, CAS NO. | | | | | |
| 3681-71-8) | | | | | |
| ALDEHYDE C11 | 4.000 | 4.000 | 4.000 | 4.000 | 4.000 |
| MOA PURE 10% | | | | | |
| DPG (2- | | | | | |
| METHYLDECANAL, | | | | | |
| CAS NO. 19009-56-4) | | | | | |
| HEXALACTONE | 5.000 | 5.000 | 5.000 | 5.000 | 5.000 |
| GAMMA 10% DPG(5- | | | | | |
| ETHYLDIHYDROFUR | | | | | |
| AN-2(3H)-ONE, CAS | | | | | |
| NO. 695-06-7) | | | | | |
| PRENYL ACETATE | 5.000 | 5.000 | 5.000 | 5.000 | 5.000 |
| 10% DPG (3- | | | | | |
| METHYLBUT-2-EN-1- | | | | | |
| YL ACETATE, CAS | | | | | |
| NO. 1191-16-8) | | | | | |
| ETHYL METHYL | 3.000 | 3.000 | 3.000 | 3.000 | 3.000 |
| BUTYRATE-2 10% | | | | | |
| DPG (ETHYL 2- | | | | | |
| METHYLBUTYRATE, | | | | | |
| CAS NO. 7452-79-1) | | | | | |
| NERYL ACETATE | 8.000 | 8.000 | 8.000 | 8.000 | 8.000 |
| 10% DPG (3,7- | | | | | |
| DIMETHYLOCTA-2,6- | | | | | |
| DIEN-1-YL | | | | | |
| ACETATE, CAS NO. | | | | | |
| 141-12-8) | | | | | |
| HEXENOL CIS-3 10% | 3.000 | 3.000 | 3.000 | 3.000 | 3.000 |
| DPG (HEX-3-EN-1- | | | | | |
| OL, CAS NO. 928-96-1) | | | | | |
| AMBROXIDE 10% | 3.000 | 3.000 | 3.000 | 3.000 | 3.000 |
| IPM | | | | | |
| ((3AR,5AS,9AS,9BR)- | | | | | |
| 3A,6,6,9A- | | | | | |
| TETRAMETHYL- | | | | | |
| DODECAHYDRONA | | | | | |
| PHTHO[2,1- | | | | | |
| B]FURAN, CAS NO. | | | | | |
| 6790-58-5) | | | | | |
| OCTAHYDRO | 3.000 | 3.000 | 3.000 | 3.000 | 3.000 |
| COUMARIN 10% | | | | | |
| DPG (OCTAHYDRO- | | | | | |
| 2H-1-BENZOPYRAN- | | | | | |
| 2-ONE, CAS NO. | | | | | |
| 4430-31-3) | | | | | |
| SANDRANOL ® ((E)- | 6.000 | 6.000 | 6.000 | 6.000 | 6.000 |
| 2-ETHYL-4-(2,2,3- | | | | | |
| TRIMETHYL-1- | | | | | |
| CYCLOPENT-3- | | | | | |
| ENYL)BUT-2-EN-1- | | | | | |
| OL, CAS NO. 28219- | | | | | |
| 61-6) | | | | | |
| AGRUMEX (2-TERT- | 20.000 | 20.000 | 20.000 | 20.000 | 20.000 |
| BUTYLCYCLOHEXYL | | | | | |
| ACETATE, CAS | | | | | |
| NO. 20298-69-5) | | | | | |
| ALDEHYDE C14 SO- | 4.000 | 4.000 | 4.000 | 4.000 | 4.000 |
| CALLED (5- | | | | | |
| HEPTYLOXOLAN-2- | | | | | |
| ONE, CAS NO. 104- | | | | | |
| 67-6) | | | | | |
| HEXYL ACETATE | 3.000 | 3.000 | 3.000 | 3.000 | 3.000 |
| (HEXYL ACETATE, | | | | | |
| CAS NO. 142-92-7) | | | | | |
| STYRALYL | 6.000 | 6.000 | 6.000 | 6.000 | 6.000 |
| ACETATE (1- | | | | | |
| PHENYLETHYL | | | | | |
| ACETATE, CAS NO. | | | | | |
| 93-92-5) | | | | | |
| BENZYL ACETATE | 5.000 | 5.000 | 5.000 | 5.000 | 5.000 |
| (CAS NO. 140-11-4) | | | | | |
| MYSORE ACETATE | 12.000 | 12.000 | 12.000 | 12.000 | 12.000 |
| (OCTAHYDRO-4,7- | | | | | |
| METHANO-1H- | | | | | |
| INDENEMETHYL | | | | | |
| ACETATE, CAS NO. | | | | | |
| 30772-69-1) | | | | | |
| LACTOJASMONE (5- | 2.000 | 2.000 | 2.000 | 2.000 | 2.000 |
| HEXYLDIHYDRO-5- | | | | | |
| METHYLFURAN- | | | | | |
| 2(3H)-ONE, CAS NO. | | | | | |
| 7011-83-8) | | | | | |
| BORNEOL | 2.000 | 2.000 | 2.000 | 2.000 | 2.000 |
| L/ISOBORNEOL | | | | | |
| 65/35 10% DPG | | | | | |
| (REACTION MASS | | | | | |
| OF DL-BORNEOL | | | | | |
| AND EXO-1,7,7- | | | | | |
| TRIMETHYLBICYCLO | | | | | |
| [2.2.1]HEPTAN-2- | | | | | |
| OL, CAS NO. NOT | | | | | |
| AVAILABLE) | | | | | |
| ALLYL | 1.000 | 1.000 | 1.000 | 1.000 | 1.000 |
| CYCLOHEXYL | | | | | |
| PROPIONATE | | | | | |
| (PROP-2-EN-1-YL 3- | | | | | |
| CYCLOHEXYLPROP | | | | | |
| ANOATE, CAS NO. | | | | | |
| 2705-87-5) | | | | | |
| LINALYL ACETATE | 24.000 | 24.000 | 24.000 | 24.000 | 24.000 |
| (1,5-DIMETHYL-1- | | | | | |
| VINYLHEX-4-EN-1- | | | | | |
| YL ACETATE, CAS | | | | | |
| NO. 115-95-7) | | | | | |
| CYCLODUMOL | 16.000 | 16.000 | 16.000 | 16.000 | 16.000 |
| ACETATE (1-(3,3- | | | | | |
| DIMETHYLCYCLOHEXYL) | | | | | |
| ETHYL | | | | | |
| ACETATE, CAS NO. | | | | | |
| 25225-10-9) | | | | | |
| FLOROSA BM/ | 35.000 | 35.000 | 35.000 | 35.000 | 35.000 |
| PYRANOL (4- | | | | | |
| METHYL-2-(2- | | | | | |
| METHYLPROPYL)OX | | | | | |
| AN-4-OL, CAS NO. | | | | | |
| 63500-71-0) | | | | | |
| PHENOXYETHYL | 400.000 | 300.000 | 300.000 | 300.000 | 300.000 |
| ALCOHOL (2- | | | | | |
| PHENOXYETHAN-1- | | | | | |
| OL, CAS NO. 122-99-6) | | | | | |
| METHYL | 0.000 | 100.000 | 0.000 | 0.000 | 0.000 |
| DIHYDROJASMONATE | | | | | |
| (CAS NO. 24851- | | | | | |
| 98-7) | | | | | |
| Product of Example 1 | 0.000 | 0.000 | 100.000 | 0.000 | 0.000 |
| (cis: trans mixture, | | | | | |
| 55:45) | | | | | |
| Product of Example | 0.000 | 0.000 | 0.000 | 100.000 | 0.000 |
| 2.1 (cis: trans mixture, | | | | | |
| 61:38) | | | | | |
| Product of Example 8 | 0.000 | 0.000 | 0.000 | 0.000 | 100.000 |
| | 1000.000 | 1000.000 | 1000.000 | 1000.000 | 1000.000 |

Perfume oils 6 to 10 were evaluated in body lotion at a concentration of 0.3%, respectively, by a panel of trained perfumers. To that purpose, the prepared lotions were olfactorily evaluated in blind against a benchmark (methyl dihydrojasmonate in perfume oil 7) and an odorless solvent (perfume oil 6).

The products of Examples 1, 2.1, and 8 enhance the creaminess of the fragrance and increase the floral character. The product of Example 1 and the product of Example 2.1 cover the odor of the body-lotion base much better than methyl dihydrojasmonate. The fragrance gets a more distinct and fresher citrus bloom in the top note. Also, this effect is stronger than with methyl dihydrojasmonate.

### Example 14: Eau de Toilette

TABLE 3

| | Perfume | Perfume | | | | |
|---|---|---|---|---|---|---|
| | oil 11 | oil 12 | Perfume | Perfume | Perfume | Perfume |
| | (not | (not | oil 13 | oil 14 | oil 15 | oil 16 |
| | according | according | (according | (according | (according | (according |
| | to | to | to | to | to | to |
| Ingredients | invention) | invention) | invention) | invention) | invention) | invention) |
| CASSIS BASE | 1.000 | 1.000 | 1.000 | 1.000 | 1.000 | 1.000 |
| 345 BB | | | | | | |
| (PURCHASED | | | | | | |
| FRAGRANCE | | | | | | |
| BASE - NO CAS | | | | | | |
| OR IUPAC | | | | | | |
| AVAILABLE) | | | | | | |
| YLANG YLANG | 1.000 | 1.000 | 1.000 | 1.000 | 1.000 | 1.000 |
| OIL (CANANGA | | | | | | |
| ODORATA OIL, | | | | | | |
| CAS NO. 8006-81-3) | | | | | | |
| LEMON OIL (CAS | 15.000 | 15.000 | 15.000 | 15.000 | 15.000 | 15.000 |
| NO. 8008-56-8) | | | | | | |
| VETIVER OIL | 3.000 | 3.000 | 3.000 | 3.000 | 3.000 | 3.000 |
| (CAS NO. 8016-96-4) | | | | | | |
| MANDARIN OIL | 10.000 | 10.000 | 10.000 | 10.000 | 10.000 | 10.000 |
| (CAS NO. 84929-38-4) | | | | | | |
| DIHYDROMYRCENOL (2,6- | 25.000 | 25.000 | 25.000 | 25.000 | 25.000 | 25.000 |
| DIMETHYLOCT-7- | | | | | | |
| EN-2-OL, CAS | | | | | | |
| NO. 18479-58-8) | | | | | | |
| HYDROXY | 10.000 | 10.000 | 10.000 | 10.000 | 10.000 | 10.000 |
| CITRONELLAL (7- | | | | | | |
| HYDROXY-3,7- | | | | | | |
| DIMETHYLOCTANAL, | | | | | | |
| CAS NO. 107-75-5) | | | | | | |
| HEXYL | 7.000 | 7.000 | 7.000 | 7.000 | 7.000 | 7.000 |
| SALICYLATE | | | | | | |
| (CAS NO. 6259-76-3) | | | | | | |
| FLOROSA (4- | 70.000 | 70.000 | 70.000 | 70.000 | 70.000 | 70.000 |
| METHYL-2-(2- | | | | | | |
| METHYLPROPYL) | | | | | | |
| OXAN-4-OL, CAS | | | | | | |
| NO. 63500-71-0) | | | | | | |
| HELIONAL (3- | 20.000 | 20.000 | 20.000 | 20.000 | 20.000 | 20.000 |
| (1,3- | | | | | | |
| BENZODIOXOL-5- | | | | | | |
| YL)-2- | | | | | | |
| METHYLPROPANAL, | | | | | | |
| CAS NO. 1205-17-0) | | | | | | |
| BERGAMOT OI | 20.000 | 20.000 | 20.000 | 20.000 | 20.000 | 20.000 |
| (CAS NO. 8007-75-8) | | | | | | |
| GERANIOL (3,7- | 7.000 | 7.000 | 7.000 | 7.000 | 7.000 | 7.000 |
| DIMETHYLOCTA- | | | | | | |
| 2,6-DIEN-1-OL, | | | | | | |
| CAS NO. 106-24-1) | | | | | | |
| AMBROXIDE | 3.000 | 3.000 | 3.000 | 3.000 | 3.000 | 3.000 |
| ((3AR,5AS,9AS,9BR)- | | | | | | |
| 3A,6,6,9A- | | | | | | |
| TETRAMETHYL- | | | | | | |
| DODECAHYDRO- | | | | | | |
| NAPHTHO[2,1- | | | | | | |
| B]FURAN, CAS | | | | | | |
| NO. 6790-58-5) | | | | | | |
| JASMIN ABS. | 2.000 | 2.000 | 2.000 | 2.000 | 2.000 | 2.000 |
| (CAS NO. 8022-96-6) | | | | | | |
| MAJANTOL ® (3- | 7.000 | 7.000 | 7.000 | 7.000 | 7.000 | 7.000 |
| (2,2-DIMETHYL-3- | | | | | | |
| HYDROXYPROPYL) | | | | | | |
| TOLUENE, CAS | | | | | | |
| NO. 103694-68-4) | | | | | | |
| GLOBALIDE ® | 30.000 | 30.000 | 30.000 | 30.000 | 30.000 | 30.000 |
| (((12E)-1- | | | | | | |
| OXACYCLOHEXADEC- | | | | | | |
| 12-EN-2-ONE; (12Z)-1- | | | | | | |
| OXACYCLOHEXADEC- | | | | | | |
| 12-EN-2-ONE; (13E)-1- | | | | | | |
| OXACYCLOHEXADEC- | | | | | | |
| 13-EN-2-ONE; (13Z)-1- | | | | | | |
| OXACYCLOHEXADEC- | | | | | | |
| 13-EN-2-ONE)), | | | | | | |
| CAS NO. 111879-80-2) | | | | | | |
| POLYSANTOL | 4.000 | 4.000 | 4.000 | 4.000 | 4.000 | 4.000 |
| ((4E)-3,3- | | | | | | |
| DIMETHYL-5-(2,2,3- | | | | | | |
| TRIMETHYLCYCLOPENT- | | | | | | |
| 3-EN-1-YL)PENT-4-EN-2-OL, | | | | | | |
| CAS NO. 107898-54-4) | | | | | | |
| RED BERRY | 5.000 | 5.000 | 5.000 | 5.000 | 5.000 | 5.000 |
| EXTRACT CO2 | | | | | | |
| (PINK PEPPER | | | | | | |
| CO2 EXTRACT, | | | | | | |
| CAS NO. 949495-68-5) | | | | | | |
| ORANGE OIL | 20.000 | 20.000 | 20.000 | 20.000 | 20.000 | 20.000 |
| SWEET (CAS NO. | | | | | | |
| 8008-57-9) | | | | | | |
| GERANYL | 5.000 | 5.000 | 5.000 | 5.000 | 5.000 | 5.000 |
| ACETATE (3,7- | | | | | | |
| DIMETHYLOCTA- | | | | | | |
| 2,6-DIEN-1-YL | | | | | | |
| ACETATE, CAS | | | | | | |
| NO. 105-87-3) | | | | | | |
| COUMARIN (2H- | 2.000 | 2.000 | 2.000 | 2.000 | 2.000 | 2.000 |
| CHROMEN-2- | | | | | | |
| ONE, CAS NO. | | | | | | |
| 91-64-5) | | | | | | |
| LINALOOL (3,7- | 55.000 | 55.000 | 55.000 | 55.000 | 55.000 | 55.000 |
| DIMETHYLOCTA- | | | | | | |
| 1,6-DIEN-3-OL, | | | | | | |
| CAS NO. 78-70-6) | | | | | | |
| LINALYL | 28.000 | 28.000 | 28.000 | 28.000 | 28.000 | 28.000 |
| ACETATE (1,5- | | | | | | |
| DIMETHYL-1- | | | | | | |
| VINYLHEX-4-EN- | | | | | | |
| 1-YL ACETATE, | | | | | | |
| CAS NO. 115-95-7) | | | | | | |
| PHENYLETHYL | 5.000 | 5.000 | 5.000 | 5.000 | 5.000 | 5.000 |
| ALCOHOL (2- | | | | | | |
| PHENYLETHANOL, | | | | | | |
| CAS NO. 60-12-8) | | | | | | |
| VANILLIN (4- | 2.000 | 2.000 | 2.000 | 2.000 | 2.000 | 2.000 |
| HYDROXY-3- | | | | | | |
| METHOXYBENZALDE- | | | | | | |
| HYDE, CAS NO. 121-33-5) | | | | | | |
| CYCLAMEN | 1.500 | 1.500 | 1.500 | 1.500 | 1.500 | 1.500 |
| ALDEHYDE (2- | | | | | | |
| METHYL-3-[4- | | | | | | |
| (PROPAN-2-YL) | | | | | | |
| PHENYL]PROPANAL, | | | | | | |
| CAS NO. 103-95-7) | | | | | | |
| ETHYL LINALOOL | 40.000 | 40.000 | 40.000 | 40.000 | 40.000 | 40.000 |
| (3,7-DIMETHYLNONA- | | | | | | |
| 1,6-DIEN-3-OL, | | | | | | |
| CAS NO. 10339-55-6) | | | | | | |
| ALLYL AMYL | 5.000 | 5.000 | 5.000 | 5.000 | 5.000 | 5.000 |
| GLYCOLATE | | | | | | |
| (ALLYL (3- | | | | | | |
| METHYLBUTOXY) | | | | | | |
| ACETATE, CAS | | | | | | |
| NO. 67634-00-8) | | | | | | |
| ISO E SUPER | 30.000 | 30.000 | 30.000 | 30.000 | 30.000 | 30.000 |
| (REACTION | | | | | | |
| MASS FROM THE ACIDIC | | | | | | |
| CYCLIZATION OF | | | | | | |
| 1-(1′,6′- | | | | | | |
| DIMETHYL-4′-(4″- | | | | | | |
| METHYLPENT-3″- | | | | | | |
| EN-1″-YL)CYCLOHEX- | | | | | | |
| 3′-EN-1′- | | | | | | |
| YL)ETHAN-1- | | | | | | |
| ONE, CAS NO. | | | | | | |
| 54464-57-2) | | | | | | |
| CALONE (7- | 2.000 | 2.000 | 2.000 | 2.000 | 2.000 | 2.000 |
| METHYL-3,4- | | | | | | |
| DIHYDRO-2H-1,5- | | | | | | |
| BENZODIOXEPIN- | | | | | | |
| 3-ONE, CAS NO. | | | | | | |
| 28940-11-6) | | | | | | |
| IONONE BETA (4-(2,6,6- | 10.000 | 10.000 | 10.000 | 10.000 | 10.000 | 10.000 |
| TRIMETHYLCYCLOHEX- | | | | | | |
| 1-EN-1-YL)BUT-3-EN-2- | | | | | | |
| ONE, CAS NO. 79-77-6) | | | | | | |
| AMBRETTOLIDE | 10.000 | 10.000 | 10.000 | 10.000 | 10.000 | 10.000 |
| (OXACYCLOHEPTADEC- | | | | | | |
| 10-EN-2-ONE, CAS NO. | | | | | | |
| 28645-51-4) | | | | | | |
| MUSCENONE (4E)-3- | 20.000 | 20.000 | 20.000 | 20.000 | 20.000 | 20.000 |
| METHYLCYCLOPENTADEC- | | | | | | |
| 4-EN-1-ONE; (5E)-3- | | | | | | |
| METHYLCYCLOPENTADEC- | | | | | | |
| 5-EN-1-ONE; (5Z)-3- | | | | | | |
| METHYLCYCLOPENTADEC- | | | | | | |
| 5-EN-1-ONE, CAS NO. | | | | | | |
| 63314-79-4) | | | | | | |
| VELVIONE (5- | 30.000 | 30.000 | 30.000 | 30.000 | 30.000 | 30.000 |
| CYCLOHEXADECEN- | | | | | | |
| 1-ONE, CAS | | | | | | |
| NO. 37609-25-9) | | | | | | |
| LILYBELLE ® 10% | 3.000 | 3.000 | 3.000 | 3.000 | 3.000 | 3.000 |
| DPG (3-[(4R)-4- | | | | | | |
| (PROPAN-2- | | | | | | |
| YL)CYCLOHEX-1- | | | | | | |
| EN-1-YL]PROPANAL, | | | | | | |
| CAS NO. | | | | | | |
| 1378867-81-2) | | | | | | |
| OSYROL 10% | 4.000 | 4.000 | 4.000 | 4.000 | 4.000 | 4.000 |
| DPG (REACTION | | | | | | |
| MASS OF (R*,R*)- | | | | | | |
| 7-METHOXY-3,7- | | | | | | |
| DIMETHYL-2- | | | | | | |
| OCTANOL AND | | | | | | |
| (R*,S*)-7- | | | | | | |
| METHOXY-3,7- | | | | | | |
| DIMETHYL-2- | | | | | | |
| OCTANOL, CAS | | | | | | |
| NO. 41890-92-0) | | | | | | |
| OSMANTHUS | 2.000 | 2.000 | 2.000 | 2.000 | 2.000 | 2.000 |
| ABS. 10% DPG | | | | | | |
| (CAS NO. 92347-21-2) | | | | | | |
| FLORALOZONE | 3.000 | 3.000 | 3.000 | 3.000 | 3.000 | 3.000 |
| 10% DPG | | | | | | |
| (REACTION | | | | | | |
| MASS OF 3-(O- | | | | | | |
| ETHYLPHENYL)-2,2- | | | | | | |
| DIMETHYLPROPIONALDE- | | | | | | |
| HYDE AND 3- | | | | | | |
| (PETHYLPHENYL)2,2- | | | | | | |
| DIMETHYLPROPIONALDE- | | | | | | |
| HYDE, CAS NO. | | | | | | |
| NOT AVAILABLE) | | | | | | |
| MEFRANAL 10% | 5.000 | 5.000 | 5.000 | 5.000 | 5.000 | 5.000 |
| DPG (3-METHYL-5- | | | | | | |
| PHENYLPENTANAL, | | | | | | |
| CAS NO. 55066-49-4) | | | | | | |
| HEXENYL | 2.000 | 2.000 | 2.000 | 2.000 | 2.000 | 2.000 |
| ACETATE CIS-3 | | | | | | |
| 10% DPG ((Z)- | | | | | | |
| HEX-3-ENYL | | | | | | |
| ACETATE, CAS | | | | | | |
| NO. 3681-71-8) | | | | | | |
| MARJORAM OIL | 4.000 | 4.000 | 4.000 | 4.000 | 4.000 | 4.000 |
| EGYPT. 10% DPG | | | | | | |
| (CAS NO. 84082-58-6) | | | | | | |
| ROSE DAMASCENA OIL | 3.000 | 3.000 | 3.000 | 3.000 | 3.000 | 3.000 |
| 10% TEC (CAS | | | | | | |
| NO. 8007-01-0) | | | | | | |
| ETHYL MALTOL | 7.000 | 7.000 | 7.000 | 7.000 | 7.000 | 7.000 |
| 1% DPG (2- | | | | | | |
| ETHYL-3- | | | | | | |
| HYDROXY-4H- | | | | | | |
| PYRAN-4-ONE, | | | | | | |
| CAS NO. 4940-11-8) | | | | | | |
| DIMETHYL BENZYL | 1.000 | 1.000 | 1.000 | 1.000 | 1.000 | 1.000 |
| CARBINYL | | | | | | |
| ACETATE 10% | | | | | | |
| DPG (1,1-DIMETHYL-2- | | | | | | |
| PHENYLETHYL | | | | | | |
| ACETATE, CAS | | | | | | |
| NO. 151-05-3) | | | | | | |
| DAMASCONE | 4.000 | 4.000 | 4.000 | 4.000 | 4.000 | 4.000 |
| ALPHA 10% DPG | | | | | | |
| (1-(2,6,6- | | | | | | |
| TRIMETHYLCYCLOHEX- | | | | | | |
| 2-EN-1-YL)BUT-2-EN-1- | | | | | | |
| ONE, CAS NO. | | | | | | |
| 24720-09-0) | | | | | | |
| BOURGEONAL | 4.000 | 4.000 | 4.000 | 4.000 | 4.000 | 4.000 |
| 10% DPG (3-(4- | | | | | | |
| TERT-BUTYLPHENYL) | | | | | | |
| PROPANAL, CAS | | | | | | |
| NO. 18127-01-0) | | | | | | |
| LEAFOVERT ® | 2.000 | 2.000 | 2.000 | 2.000 | 2.000 | 2.000 |
| 10% DPG ((3Z)- | | | | | | |
| HEX-3-EN-1-YL | | | | | | |
| METHYL CARBONATE, | | | | | | |
| CAS NO. 67633-96-9) | | | | | | |
| ALDEHYDE C14 | 3.000 | 3.000 | 3.000 | 3.000 | 3.000 | 3.000 |
| SO-CALLED | | | | | | |
| EXTRA 10% DPG | | | | | | |
| (5-HEPTYLOXOLAN- | | | | | | |
| 2-ONE, CAS NO. | | | | | | |
| 104-67-6) | | | | | | |
| JASMONE CIS | 8.000 | 8.000 | 8.000 | 8.000 | 8.000 | 8.000 |
| 10% DPG (3- | | | | | | |
| METHYL-2-PENT-2- | | | | | | |
| ENYLCYCLOPENT- | | | | | | |
| 2-ENONE, CAS | | | | | | |
| NO. 488-10-8) | | | | | | |
| ETHYL VANILLIN | 4.000 | 4.000 | 4.000 | 4.000 | 4.000 | 4.000 |
| 10% DPG (3- | | | | | | |
| ETHOXY-4- | | | | | | |
| HYDROXYBENZALDE- | | | | | | |
| HYDE, CAS | | | | | | |
| NO. 121-32-4) | | | | | | |
| DAMASCENONE | 1.500 | 1.500 | 1.500 | 1.500 | 1.500 | 1.500 |
| 10% DPG (1-(2,6,6- | | | | | | |
| TRIMETHYLCYCLOHEXA- | | | | | | |
| 1,3-DIEN-1-YL) | | | | | | |
| BUT-2-EN-1-ONE, | | | | | | |
| CAS NO. 23726-93-4) | | | | | | |
| HEXENOL CIS-3 | 2.000 | 2.000 | 2.000 | 2.000 | 2.000 | 2.000 |
| 10% DPG (HEX-3- | | | | | | |
| EN-1-OL, CAS | | | | | | |
| NO. 928-96-1) | | | | | | |
| AURELIONE ® | 70.000 | 70.000 | 70.000 | 70.000 | 70.000 | 70.000 |
| (CYCLOHEXADECANONE, | | | | | | |
| CAS NO. 2550-59-6) | | | | | | |
| HEXENYL | 25.000 | 25.000 | 25.000 | 25.000 | 25.000 | 25.000 |
| SALICYLATE CIS-3 | | | | | | |
| (HEX-3-EN-1-YL | | | | | | |
| SALICYLATE, | | | | | | |
| CAS NO. 65405-77-8) | | | | | | |
| VETIVERYL | 4.000 | 4.000 | 4.000 | 4.000 | 4.000 | 4.000 |
| ACETATE EXTRA | | | | | | |
| (REACTION | | | | | | |
| MASS FROM THE | | | | | | |
| ACETYLATION | | | | | | |
| OF VETIVER OIL, | | | | | | |
| CAS NO. 68917-34-0) | | | | | | |
| STYRALYL | 2.000 | 2.000 | 2.000 | 2.000 | 2.000 | 2.000 |
| ACETATE (1- | | | | | | |
| PHENYLETHYL | | | | | | |
| ACETATE, CAS | | | | | | |
| NO. 93-92-5) | | | | | | |
| PATCHOULI OIL | 15.000 | 15.000 | 15.000 | 15.000 | 15.000 | 15.000 |
| (CAS NO. 8014-09-3) | | | | | | |
| ECOMUSK R (2-[1-(3,3- | 20.000 | 20.000 | 20.000 | 20.000 | 20.000 | 20.000 |
| DIMETHYLCYCLOHEXYL) | | | | | | |
| ETHOXY]-2-OXOETHYL | | | | | | |
| PROPANOATE, | | | | | | |
| CAS NO. 236391-76-7) | | | | | | |
| BENZYL SALICYLATE | 40.000 | 40.000 | 40.000 | 40.000 | 40.000 | 40.000 |
| (CAS NO. 118-58-1) | | | | | | |
| NEO HELIOPAN ® | 6.000 | 6.000 | 6.000 | 6.000 | 6.000 | 6.000 |
| AV (2-ETHYLHEXYL 4- | | | | | | |
| METHOXYCINNAMATE, | | | | | | |
| CAS NO. 5466-77-3) | | | | | | |
| DIPROPYLENE | 250.000 | 0.000 | 0.000 | 0.000 | 0.000 | 0.000 |
| GLYCOL (1,1- | | | | | | |
| OXYDI-2-PROPANOL, | | | | | | |
| CAS NO. 25265-71-8) | | | | | | |
| METHYL | 0.000 | 250.000 | 0.000 | 0.000 | 0.000 | 0.000 |
| DIHYDROJASMONATE | | | | | | |
| (CAS NO. 24851-98-7) | | | | | | |
| Product of Example 1 | 0.000 | 0.000 | 250.000 | 0.000 | 0.000 | 0.000 |
| (cis:trans mixture, 55:45) | | | | | | |
| Product of Example 2.1 | 0.000 | 0.000 | 0.000 | 250.000 | 0.000 | 0.000 |
| (cis:trans mixture, 61:38) | | | | | | |
| Product of Example 8 | 0.000 | 0.000 | 0.000 | 0.000 | 250.000 | 0.000 |
| Product of Example 10 | 0.000 | 0.000 | 0.000 | 0.000 | 0.000 | 250.000 |
| | 1000.000 | 1000.000 | 1000.000 | 1000.000 | 1000.000 | 1000.000 |

Perfume oils 11 to 16 were evaluated in eau de toilette at a concentration of 10%, respectively, by a panel of trained perfumers. The samples of the eau de toilette were olfactorily evaluated in blind by the panel against a benchmark (methyl dihydrojasmonate in perfume oil 12) and an odorless solvent (perfume oil 11).

The fragrance is much stronger with the product of Examples 1, 2.1, 8, and 10, respectively, or methyl dihydrojasmonate compared to the version with only solvent. The products of Examples 1, 2.1, 8, and 10 work as a key floral note in the fragrance and make it more powdery and transparent. The fragrance has more of a fine fragrance appeal. The product of Example 2.1 has a particularly positive effect on the floral jasmine note and improves the radiance of the fragrance. Additionally, this version is the most similar one compared to the version with methyl dihydrojasmonate even if methyl dihydrojasmonate is more powerful.

### Example 15: Special Perfumery effects of 4-(allyloxymethyl)-2-hexyl-1,3-dioxolane (Product of Example 2.1, cis:trans Mixture, 61:38) with Important Perfumery Ingredients

The product of Example 2.1 (cis:trans mixture, 61:38) was added to the respective pure fragrance substance (cf. column “CAS No.” and “Name” in Table 4 below) in a percentage amount as indicated in Table 4 below (cf. column “Dosage of product of Example 2.1”). The mixture of pure fragrance substance and product of Example 2.1 was tested against pure fragrance substance.

Only Ambroxide, which is solid at room temperature, was dissolved in isopropylmyristate (IPM) to produce a 10% solution. Then, the product of Example 2.1 was added in a percentage amount as indicated in Table 4 below (cf. column “Dosage of product of Example 2.1”) to said solution of Ambroxide in IPM. The mixture of the solution of Ambroxide in IPM and the product of Example 2.1 was tested against the solution of Ambroxide in IPM.

A panel of at least two trained perfumers evaluated the samples on smelling strips immediately after dipping the smelling strips into the respective sample.

TABLE 4

| | | | Dosage of | |
|---|---|---|---|---|
| | | | product of | |
| | | | Example | |
| Nr .: | CAS No. | Name | 2.1 | Olfactive effect |
| 1 | 20298-69-5 | Agrumex | 3% | More tobacco |
| | | | | Drier in the direction |
| | | | | of Damascone Delta |
| 2 | 7452-79-1 | Ethylmethyl-2- | 2% | Rounder and more in |
| | | butyrate | | the direction of apple |
| 3 | 123-92-2 | Isoamyl Acetate | 4% | Prenyl acetate effect |
| | | | | More cooling |
| 4 | 10094-34-5 | Dimethyl Benzyl | 2% | Marmelade effect |
| | | Carbinyl Butyrate | | Significantly shifts the |
| | | | | effect into dimethyl |
| | | | | benzyl carbinyl |
| | | | | acetate |
| 5 | 142-92-7 | Hexyl Acetate | 1% | Provides the juicy and |
| | | | | watery facet of pear |
| | | | | Less green-grassy |
| 6 | 2705-87-5 | Allyl Cyclohexyl | 1% | Enhances the |
| | | Propionate | | pineapple juiciness |
| | | | | Less cinnamate and |
| | | | | less technical in |
| | | | | appeal |
| 7 | 18479-58-8 | Dihydro Myrcenol | 10% | Boosts the linalool |
| | | | | effect |
| | | | | Stronger in the |
| | | | | coriander note |
| 8 | 10339-55-6 | Ethyl Linalool | 1% | Pushes the fruity- |
| | | | | blueberry facet |
| 9 | 1205-17-0 | Helional | 2% | Stronger anisic note |
| | | | | Shifts the note in the |
| | | | | direction of Canthoxal |
| 10 | 103-95-7 | Cyclamen Aldehyde | 2% | Pushes the ozonic |
| | | | | apsects |
| | | | | Less salty |
| | | | | Enhances the floralcy |
| | | | | and the cyclamen |
| | | | | note |
| 11 | 488-10-8 | cis-Jasmone | 1% | Enhances the floral- |
| | | | | jasmine note |
| | | | | Hay effect |
| | | | | Reduces the celery |
| | | | | facets |
| 12 | 140-11-4 | Benzyl Acetate | 6% | Stronger in the floral- |
| | | | | jasmine note |
| | | | | Less technical |
| 13 | 24851-98-7 | Methyl | 10% | Pushes the tea note |
| | | dihydrojasmonate | | and provides more |
| | | | | body to the |
| | | | | composition |
| | | | | Reduces the fruitiness |
| 14 | 134-20-3 | Methyl Anthranilate | 10% | Provides more volume |
| | | | | and reduces the |
| | | | | narcotic effect of the |
| | | | | anthranilate |
| | | | | Enhances the orange |
| | | | | flower note |
| 15 | 1335-46-2 | Methyl Ionone 70 | 5% | Twists the |
| | | | | composition more into |
| | | | | a floral-violet direction |
| | | | | and reduces the orris |
| | | | | note |
| 16 | 79-77-6 | beta-Ionone | 2% | Increases the |
| | | | | woodiness of the |
| | | | | ingredient |
| 17 | 106-24-1 | Geraniol Supra | 5% | Pronounced fruity and |
| | | | | citrus facets |
| 18 | 23726-93-4 | Damascenone | 6% | Enhances the tobacco |
| | | | | note |
| | | | | Provides creaminess |
| | | | | Stresses the fruity- |
| | | | | plum facets |
| 19 | 60-12-8 | Phenylethyl Alcohol | 10% | Provides more rosy |
| | | | | naturalness |
| | | | | Increases the leathery |
| | | | | and honey facets |
| | | | | Provides more |
| | | | | strength |
| 20 | 118-58-1 | Benzyl Salicylate | 2% | Provides more |
| | | | | strength |
| | | | | Increases solar |
| | | | | aspects and white |
| | | | | floral character |
| 21 | 6259-76-3 | 3-cis-Hexyl Salicylate | 3% | Increases the floralcy |
| | | | | and reduces the pear- |
| | | | | skin note |
| 22 | 6790-58-5 | Ambroxide | 5% | Softer |
| | | | | More radiance |
| | | | | Increases the |
| | | | | powdery cedarwood |
| | | | | effect |
| 23 | 54464-57-2 | Iso E Super | 5% | Increases the |
| | | | | cedarwood note and |
| | | | | makes it more |
| | | | | powdery and creamy |
| 24 | 77-54-3 | Cedryl Acetate | 2% | Provides an |
| | | | | enhanced leathery |
| | | | | effect |
| 25 | 28219-61-6 | Sandranol ® | 5% | Increases the milky |
| | | | | sandalwood effect |
| | | | | Enhances the rosy |
| | | | | facet |
| 26 | 107898-54-4 | Polysantol ® | 8% | Twists the rosy facets |
| | | | | to a more petaly |
| | | | | character |
| | | | | Increases the |
| | | | | milkysandalwood note |
| | | | | Increases the fruity- |
| | | | | raspberry aspects |
| 27 | 68917-34-0 | Vetiveryl Acetate | 3% | Brings a stronger |
| | | Extra | | vetiver effect and |
| | | | | reduces the grapefruit |
| | | | | tonality |
| 28 | 28645-51-4 | Ambrettolide | 10% | Provides a natural |
| | | | | ambrette seeds effect |
| | | | | and increases the |
| | | | | radiance |
| 29 | 105-95-3 | Ethylene Brassylate | 5% | Enhances the fruity |
| | | | | note |
| | | | | Provides an increased |
| | | | | cosmetic creamy |
| | | | | effect |
| 30 | 111879-80-2 | Globalide ® | 5% | Increases the |
| | | | | radiance and reduces |
| | | | | metallic facets |
| | | | | Provides a fur effect |
| 31 | 63314-79-4 | Muscenone ® | 5% | Much enhances the |
| | | | | power and potency |
| | | | | Provides more earthy |
| | | | | and animalic |
| | | | | character |

1-15. (canceled)

16. A compound of formula (I): 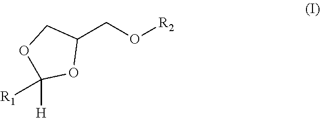
wherein
R₁ is selected from the group consisting of n-pentyl, n-hexyl, and n-heptyl, and
R₂ is selected from the group consisting of methyl, ethyl, n-propyl, isopropyl, allyl,

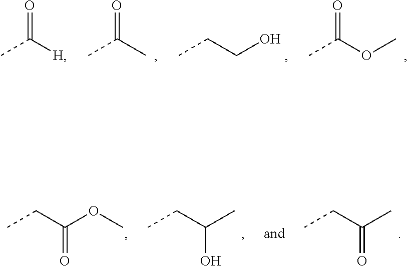

17. The compound of claim 16, wherein
R₁ is either n-hexyl or n-heptyl; and/or
R₂ is selected from the group consisting of methyl, ethyl, n-propyl, allyl,

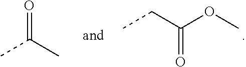

18. The compound of claim 16, wherein the compound is selected from the group consisting of:

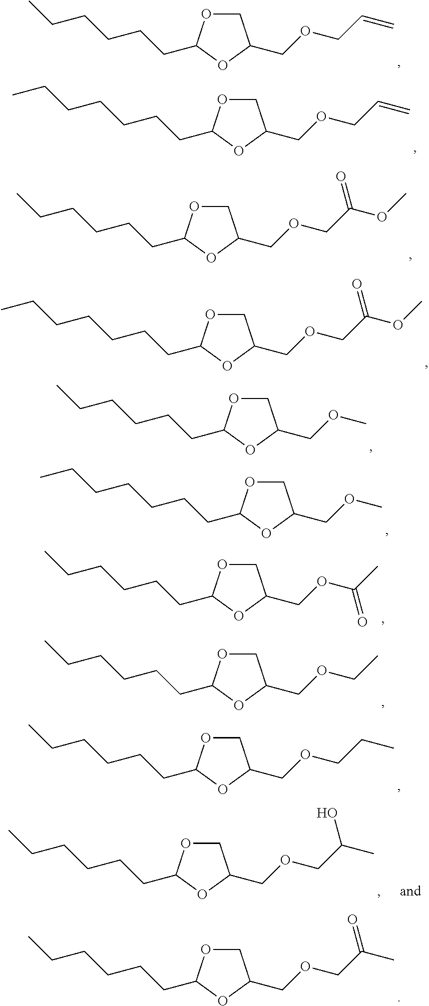

19. The compound of claim 16, wherein the compound is a compound of formula (Ia) or a compound of formula (Ib): 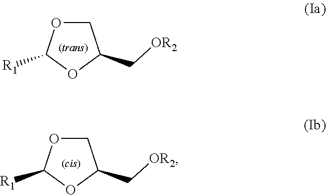
wherein
R₁ is selected from the group consisting of n-pentyl, n-hexyl and n-heptyl; and
R₂ is selected from the group consisting of methyl, ethyl, n-propyl, isopropyl, allyl,

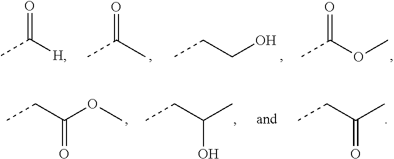

20. The compound of claim 19 wherein the compound is a compound of formula (Ia).

21. A mixture comprising a compound of formula (Ia) and a compound of formula (Ib) according to claim 19, wherein the compound of formula (Ia) and the compound of formula (Ib) are compounds with identical constitutional formulae.

22. The mixture of claim 21, wherein the compound of formula (Ia) and the compound of formula (Ib) are in a weight ratio of from 9:1 to 99:1.

23. A method for manufacturing a compound of formula (I) as defined in claim 16 comprising:
(i) reacting an aldehyde of formula (II): 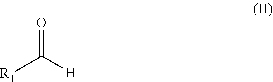
wherein R₁ is selected from the group consisting of n-pentyl, n-hexyl, and n-heptyl,
with a compound of formula (III): 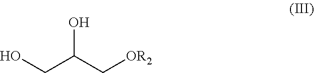
wherein R₂ is selected from the group consisting of methyl, ethyl, n-propyl, isopropyl, allyl,

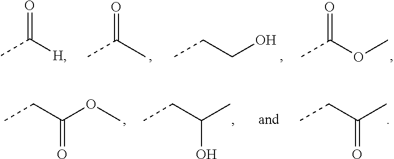

24. A method for manufacturing a compound of formula (I) as defined in claim 16 or a compound of formula (VIII): 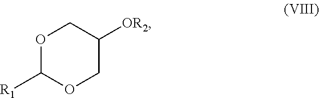
wherein
R₁ is selected from the group consisting of n-pentyl, n-hexyl, and n-heptyl; and
R₂ is selected from the group consisting of methyl, ethyl, n-propyl, isopropyl, allyl, 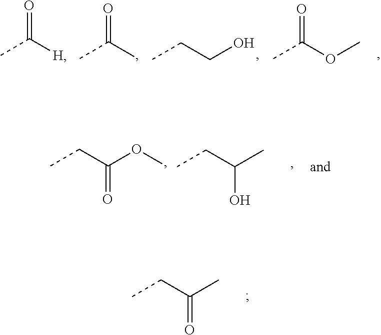
the method comprising:
(i) reacting an aldehyde of formula (II): 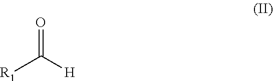
wherein R₁ of the compound of formula (II) is selected from the group consisting of n-pentyl, n-hexyl and n-heptyl,
with glycerol 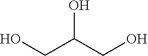
to obtain a compound of formula (IV) or a compound of formula (V): 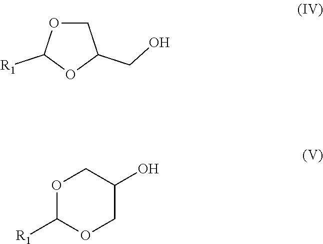
wherein R₁ of the compound of formula (IV) and formula (V) is selected from the group consisting of n-pentyl, n-hexyl, and n-heptyl; and
(ii) reacting the compound of formula (IV) or formula (V) with a compound of formula (VI), 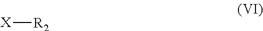
wherein X is selected from the group consisting of —Cl, —Br, and —I, and R₂ is selected from the group consisting of methyl, ethyl, n-propyl, isopropyl, allyl, 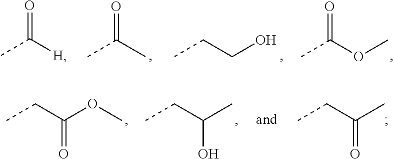
or a compound of formula (VII) 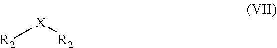
wherein X is —O— and R₂ is selected from the group consisting of 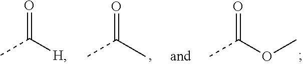
(iii) optionally, hydrogenating one or more carbon-carbon double bonds, if present, to carbon-carbon single bonds.

25. The method of claim 24, wherein R1 of the compound of formula (II) is selected from the group consisting of n-hexyl and n-heptyl, and R1 of the compound of formula (IV) and formula (V) is selected from the group consisting of n-hexyl and n-heptyl.

26. A compound of formula (VIII): 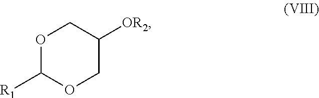
wherein
R₁ is selected from the group consisting of n-pentyl, n-hexyl, and n-heptyl; and
R₂ is selected from the group consisting of methyl, ethyl, n-propyl, isopropyl, allyl,

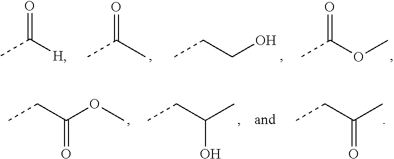

27. A method of providing or enhancing a transparent soft white-floral jasminic note in a product, the method comprising:
(a) providing a product; and
(b) incorporating into the product one or more compounds of formula (I) according to claim 16 in an amount sufficient to provide or enhance the transparent soft white-floral jasminic note.

28. A method of providing or enhancing a transparent soft white-floral jasminic note in a product, the method comprising:
(a) providing a product; and
(b) incorporating into the product one or more compounds of formula (I) according to claim 26 in an amount sufficient to provide or enhance the transparent soft white-floral jasminic note.

29. A fragrance or aroma composition comprising one or more compounds of claim 16 and optionally, one or more additional fragrance or aroma substances.

30. A fragrance or aroma composition comprising one or more compounds of claim 26 and optionally, one or more additional fragrance or aroma substances.

31. A product comprising one or more compounds of claim 16 in a sensorially effective amount.

32. A product comprising one or more compounds of claim 26 in a sensorially effective amount.

33. A method for making the product of claim 31 comprising incorporating a sensorially effective amount of the one or more compounds into the product.

34. A method for making the product of claim 32 comprising incorporating a sensorially effective amount of the one or more compounds into the product.
